(19) United States
(12) Patent Application Publication
(10) Pub. No.: US 2004/0091529 A1
(43) Pub. Date: May 13, 2004

(54) Methods and dosage forms for increasing solubility of drug compositions for controlled delivery

(76) Inventors: David Edgren, Los Altos, CA (US); Patrick S.L. Wong, Burlingame, CA (US); Frank Jao, San Jose, CA (US); Robert Skluzacek, Newark, CA (US); Shu Li, Union City, CA (US); Andrew Lam, South San Francisco, CA (US); Gurdish Bhatti, Fremont, CA (US); Shaoling Li, Sunnyvale, CA (US); Atul Ayer, Palo Alto, CA (US); Winnie To, Santa Clara, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/606,575

(22) Filed: Jun. 26, 2003

Related U.S. Application Data
(60) Provisional application No. 60/392,128, filed on Jun. 26, 2002.

(51) Int. Cl. A61K 9/22

(52) U.S. Cl. 424/468

(57) ABSTRACT

Dosage forms and devices for enhancing controlled delivery of pharmaceutical agents by use of a drug core composition that increases the solubility of the pharmaceutical agent are described. The present invention provides a means of delivering high doses of lowly soluble drug in oral drug delivery systems that are convenient to swallow, for once-a-day administration.

### CROSS REFERENCE TO RELATED APPLICATIONS

This application claims benefit, under 35 USC 119(e), to U.S. provisional patent application No. 60/392,128, filed Jun. 26, 2002, which is incorporated herein by reference.

### FIELD OF THE INVENTION

This invention pertains to the controlled delivery of pharmaceutical agents and methods, dosage forms and devices thereof. In particular, the invention is directed to methods, dosage forms and devices for enhancing controlled delivery of pharmaceutical agents by use of a composition that increases the solubility of the pharmaceutical agent. The present invention provides a means for delivering high doses of lowly soluble drug in solid dosage form systems that are convenient to swallow.

### BACKGROUND OF THE INVENTION

The art is replete with descriptions of dosage forms for the controlled release of pharmaceutical agents. While a variety of sustained release dosage forms for delivering certain drugs may be known, not every drug may be suitably delivered from those dosage forms because of solubility, metabolic processes, absorption and other physical, chemical and physiological parameters that may be unique to the drug and the mode of delivery.

Similarly, dosage forms that incorporate lowly soluble drug, including high drug loading for the dosage form, provide a major challenge for controlled release delivery technology. As such, systems tend to be of such large size that patients are unwilling or unable to swallow them.

Devices in which a drug composition is delivered as a slurry, suspension or solution from a small exit orifice by the action of an expandable layer are described in U.S. Pat. Nos. 5,633,011; 5,190,765; 5,252,338; 5,620,705; 4,931,285; 5,006,346; 5,024,842; and 5,160,743. Typical devices include a tablet comprising an expandable push layer and a drug layer, which tablet is surrounded by a semipermeable membrane having a delivery orifice. In certain instances, the tablet is provided with a subcoat to delay release of the drug composition to the environment of use.

Devices in which a drug composition is delivered in a dry state from a large exit orifice by the action of an expandable layer are described in U.S. Pat. Nos. 4,892,778, 4,915,949 and 4,940,465 and 5,023,088. Those references describe a dispenser for delivering a beneficial agent to an environment of use that includes a semipermeable wall containing a layer of expandable material that pushes a dry drug layer composition out of the compartment formed by the wall. The exit orifice in the device is substantially the same diameter as the inner diameter of the compartment formed by the wall. In such devices, a substantial area of the drug layer composition is, exposed to the environment of use leading to release performance that can be subject to the stirring conditions in such environment.

Other similar devices have delivered drug by expelling discrete drug containing tablets at a controlled rate over time. U.S. Pat. Nos. 5,938,654; 4,957,494; 5,023,088; 5,110,597; 5,340,590; 4,824,675; and 5,391,381.

Other devices attempt to deliver low solubility drugs by incorporating liquid drug formulations that are released at a controlled rate over time. These devices are disclosed in U.S. Pat. Nos. 4,111,201; 5,324,280; 5,413,672; 6,174,547. However, such liquid osmotic delivery systems are limited in the concentration of drug in the liquid formulation and hence, the drug loading available, leading to delivery systems that can be of an unacceptably large size.

Still other delivery systems utilize a liquid carrier to deliver tiny time pills suspended within the liquid carrier. Such devices are disclosed U.S. Pat. No. 4,853,229; 4,961,932. These suspensions require that the therapeutic dose of pharmaceutical agent be dispensed by volume with measuring devices such as graduated cylinders or measuring spoons, a dispensing process that can be messy and inconvenient for the patient to administer.

While dosage forms delivering the drug composition to the environment of use in the dry state through a large delivery orifice may provide suitable release of drug over a prolonged period of time, the exposure of the drug layer to the variably turbulent fluid environment of use such as the upper gastrointestinal tract may result in agitation-dependent release of drug that in some circumstances is difficult to control. Moreover, such dosage forms delivering in the dry state into a semisolid environment lacking sufficient volumes of bulk water such as in the lower colonic environment of the gastrointestinal tract may have difficulty liberating the dry dispensed drug composition into the environment as the high solids content composition tends to adhere to the dosage form at the site of the large orifice. Accordingly, it may be advantageous to release the drug as a well-hydrated slurry or suspension that may be metered by control of rate of expansion of the push layer and in combination with the smaller size of the exit orifice in the dosage form to minimize effects of localized stirring conditions on delivery performance as in accordance with this invention.

The dosage forms described above deliver therapeutic agents at an approximately zero order rate of release. Recently, dosage forms have been disclosed for delivering certain drugs at approximately ascending rates of release such as ALZA Corporation's Concerta® methylphenidate product. PCT Published Application Nos. U.S. Ser. No. 99/11920 (WO 9/62496); U.S. Ser. No. 97/13816 (WO 98/06380); and U.S. Ser. No. 97/16599 (WO 98/14168). Such disclosed dosage forms involve the use of multiple drug layers with sequentially increasing concentrations of drug in each drug layer to produce the increasing delivery rate of drug over time. While such multi-layer tablet constructions represent a significant advancement to the art, these devices also have limited capability of delivering lowly soluble pharmaceutical agents, particularly those associated with relatively large doses of such agents, in a size that is acceptable for patients to swallow.

Thus, there remains a critical need for a means to deliver high doses of lowly soluble drug compounds at various delivery patterns that are convenient and feasible for patients in need to swallow. The need includes effective dosing methods, dosage forms and devices that will permit the controlled release of the drug compounds over a prolonged period of time by increasing the solubility of the active agent in order to increase the time between dosing, preferably twice a day and most preferrably to obtain a once-a-day dosing regimen. Such dosage forms should preferably have the option of delivering at an approximately zero order rate of release, ascending or other hybrid delivery rate pattern appropriate for the therapeutic agent being delivered.

### SUMMARY OF THE INVENTION

The present invention unexpectedly provides a drug core composition for both a dosage form and method for controlled delivery of high doses of lowly soluble drug compounds over an extended period of time, preferably providing once-a-day administration. This is accomplished through the use of three primary components in the drug core composition: a therapeutic agent, a structural polymer carrier and a drug solubilizing surfactant.

The present invention is directed to a novel drug core composition for a dosage form to provide once-a-day administration with therapeutic effects over 24 hours utilizing a single convenient oral dosage form. The dosage form releases a therapeutic agent for up to about 24 hours for once-a-day administration using a drug core composition that releases drug at a controlled rate.

The present invention is capable of being adapted to release at rates ranging from zero order to ascending, and other hybrids, depending upon the type and concentration of drug and upon the type and concentration of solubilizing surfactant.

The present invention can further be applied to both osmotic delivery systems and to erodible matrix tablets.

The drug core composition of the present invention may further allow the bioavailability of the therapeutic agent to be enhanced through increased absorption of lowly soluble drugs in the gastrointestinal tract, especially in the colonic region, that otherwise would not be absorbed due to the lack of sufficient bulk water to sufficiently solubilize the drug. The drug core composition may further provide permeability enhancement of the drug through mucosal lining of the gastrointestinal tract by the action of the surfactant on these biological membranes.

The present invention may be incorporated into a semipermeable membrane enveloping a bi-layer or multi-layer core containing at least a first drug core composition layer, containing a therapeutic agent and excipients, and a second expandable layer referred to as the push layer containing osmotic agents and no therapeutic agent. An orifice is drilled through the membrane on the drug-layer end of the tablet for allowing release of the active agent to the environment.

In the aqueous environment of the gastrointestinal (GI) tract, water is imbibed through the membrane at a controlled rate. This causes the push layer to swell and the drug core composition layer(s) to hydrate and form viscous, but deformable, masses. The push layer expands against the drug layer, which is pushed out through the orifice. The drug layer composition exits the system through the orifice in the membrane over prolonged periods of time as water from the gastrointestinal tract is imbibed into the delivery system. At the completion of drug release, the biologically inert components of the delivery system are eliminated as a tablet shell.

The present invention may also be incorporated into a matrix tablet delivery system containing at least a first drug core composition layer, containing a therapeutic agent, a structural polymer carrier, and a solubilizing surfactant.

In one aspect, the present invention comprises a drug core composition for a sustained release dosage form adapted to release over a prolonged period of time at a controlled rate of release.

In another aspect, the invention comprises a method of identifying the appropriate surfactant type for pairing with a particular drug type to produce a dosage form having a drug core composition adapted to release the compound at a controlled rate of release over a prolonged period of time.

In yet another aspect, the invention comprises a method of treating a condition in a subject responsive to administration of a therapeutic agent, which comprises orally administering to the subject a dosage form having a drug core composition adapted to release the compound at a controlled rate of release over a prolonged period of time. Preferably, the dosage form is administered orally, once a day.

In still another aspect, the invention comprises a drug core composition for a dosage form comprising a wall defining a compartment, the wall having an exit orifice formed or formable therein and at least a portion of the wall being semipermeable; an expandable layer located within the compartment remote from the exit orifice and in fluid communication with the semipermeable portion of the wall; and at least one drug core composition layer located within the compartment adjacent the exit orifice, the drug layer comprising a therapeutic agent, a structural polymer carrier and a surfactant.

The prior art did not appreciate that high doses of lowly soluble drugs can be made into a single controlled release dosage form or into a solid therapeutic composition as claimed herein that provides efficacious therapy over 24 hours with once-a-day administration over 24 hours. The prior art did not appreciate that a solid dosage form and a therapeutic composition can be made available comprising a structural polymer carrier and a solid surfactant.

The prior art does not make obvious a drug core composition for a solid dosage form formulated with a structural polymer carrier and a surfactant. It is well known, for example, that surfactants can be used in liquid drug delivery systems as wetting agents, drug solubilizers, meltable carriers, oily liquid fills in gel capsules for oral administration, parenteral liquids for injection, ophthalmic drops, topical ointments, salves, lotions, and creams, suppositiories, and in pulmonary and nasal sprays. By their amphipathic molecular structure comprising opposing polar hydrophilic and non-polar hydrophobic moieties with opposite physical and chemical properties, surfactants are well known to have poor cohesive properties. Accordingly, surfactants have been limited to the above applications because at room temperature, such surfactants are in the physical form of liquids, pastes, or brittle solids which physical forms and properties are widely recognized as unacceptable for use as components in compressed solid tablets sufficiently durable for manufacture and practical use. These physical properties lead away from the use of surfactants in solid dosage forms making their embodiment in the present invention unobvious.

The drug core composition of the present invention embodies a combination of surfactant and structural polymer which structural polymer is present to provide a dual role of imparting structural integrity to the solid drug core in the dry state and of providing structural viscosity in the wet state during the operation of the dosage form. The structural viscosity develops as a result of the formation of a functional hydrogel while the delivery system is in operation. The structural polymer comprises a hydrophilic polar polymer that freely interacts with polar molecules of water to form the structurally viscous mass bearing sufficient viscosity necessary to effectively suspend and conduct the dispersed and dissolved drug as a pumpable mass from the dosage form. The formation of such a hydrogel requires extensive hydrogen bonding with water molecules entering the delivery system from the environment of use. It is well known, however, that surfactants lower the attractive forces of hydrogen bonding that water molecules have for each other which surfactant property directs away from the use of surfactants in combination with hydrogel structural polymers that require interaction with these polar water molecules to form the three-dimensional structurally viscous mass.

The above presentation dictates the critical need for a drug core composition for a solid pharmaceutical dosage form and for a therapeutic composition that overcomes the shortcomings of conventional solid osmotic dosage forms and controlled release matrix forms, including tablets and capsules. These conventional dosage forms do not provide for optimal dose-regulated drug therapy over an extended period of time with high doses of lowly soluble drugs.

Therapeutic agents in high doses having low solubility are delivered by the prior art two or more times a day and with multiple divided dosage forms, which does not lend itself to controlled and sustained therapy with once-a-day administration of a single dosage form. This prior-art pattern of drug administration indicates the need for a dosage form and for a therapeutic composition that can administer high doses of low solubility therapeutic agents in a rate-controlled dose over an extended period of time to provide constant therapy, and eliminate multiple dosing of the prior art.

### BRIEF DESCRIPTION OF THE FIGURES

The following figures are not drawn to scale, and are set forth to illustrate various embodiments of the invention.

FIG. 1 illustrates one embodiment of a dosage form of this invention, illustrating the dosage form prior to administration to a subject.

FIG. 2 illustrates the dosage form of FIG. 1 in opened section, depicting a dosage form of the invention comprising an internally housed, pharmaceutically acceptable therapeutic composition.

FIG. 3 illustrates an opened view of drawing FIG. 1, illustrating a dosage form internally comprising a therapeutic composition and a separate and contacting displacement composition comprising means for pushing the therapeutic composition from the dosage form.

FIG. 4 illustrates a dosage form provided by this invention, which further includes an instant-release external overcoat of a therapeutic composition on the dosage form.

FIG. 5 illustrates an opened view of a dosage form of the present invention illustrating a therapeutic composition comprising two drug layer compositions in parallel arrangement and a separate and contacting displacement composition comprising means for pushing the therapeutic composition from the dosage form.

FIG. 6 illustrates of the solubility of a pharmaceutical active agent in aqueous solutions of surfactants. The plots in this figure represent the method of determining the appropriate surfactant for use with a particular pharmaceutical active agent by measuring the effect of different concentrations of surfactants and of different types of surfactants on drug solubility.

FIGS. 7 through 11 illustrate release patterns of a lowly soluble pharmaceutical active agent from osmotic delivery systems formulated with a single solubilizing surfactant in the drug composition and a structural polymer wherein each system is formulated with relatively high doses of the agent, a single drug layer and a displacement layer.

FIGS. 12 and 13 illustrate release patterns of a lowly soluble pharmaceutical active agent as released from osmotic delivery systems formulated with a binary blend of solubilizing surfactant in the drug composition and a structural polymer wherein each system is formulated with relatively high doses of the agent in a single drug layer and a displacement layer.

FIG. 14 illustrates a release a pattern of a lowly soluble pharmaceutical active agent as released from osmotic delivery systems formulated with a solubilizing surfactant in the drug composition and a structural polymer wherein each system is formulated with relatively high doses of the agent in two separate drug layers and a displacement layer.

In the drawing figures and specification, like parts in related figures are identified by like numbers. The terms appearing earlier in the specification and in the description of the drawing figures, as well as embodiments thereof, are further described elsewhere in the disclosure.

### DETAILED DESCRIPTION OF THE INVENTION

The present invention is best understood by reference to the following definitions, the drawings and exemplary disclosure provided herein.

Definitions

By “dosage form” is meant a pharmaceutical composition or device comprising an active pharmaceutical agent, such as topiramate or a pharmaceutically-acceptable acid addition salt thereof, a structural polymer, a solubilizing surfactant and the composition or device optionally containing inactive ingredients, i.e., pharmaceutically acceptable excipients such as disintegrants, binders, diluents, lubricants, stabilizers, antioxidants, osmotic agents, colorants, plasticizers, coatings and the like, that are used to manufacture and deliver active pharmaceutical agents.

By “active agent”, “drug”, or “therapeutic agent” is meant an agent, drug, or compound having therapeutic characteristics or a pharmaceutically-acceptable acid addition salt thereof.

By “pharmaceutically-acceptable acid addition salt” or “pharmaceutically acceptable salt”, which are used interchangeably herein, are meant those salts in which the anion does not contribute significantly to the toxicity or pharmacological activity of the salt, and, as such, they are the pharmacological equivalents of the bases of the compound. Examples of pharmaceutically acceptable acids that are useful for the purposes of salt formation include but are not limited to hydrochloric, hydrobromic, hydroiodic, citric, succinic, tartaric, maleic, acetic, benzoic, mandelic, phosphoric, nitric, mucic, isethionic, palmitic, and others.

By “lowly soluble” and “low solubility” is meant that the neat therapeutic agent in the absence of solubilizing surfactants exhibits solubility in water of no more than 100 milligrams per milliliter. Aqueous solubility is determined by adding the therapeutic agent to stirred or agitated water maintained in a constant temperature bath at a temperature of 37 degrees centigrade until equilibrium is established betweenthe dissolved and undissolved states and the concentration of dissolved drug is constant. The resulting solution saturated with active agent is then filtered, typically under pressure through a 0.8-micron Millipore filter, and the concentration in solution is measured by any appropriate analytical method including gravimetric, ultraviolet spectrophometry, chromatography, and the like.

By “sustained release” is meant predetermined continuous release of active agent to an environment over a prolonged period.

The expressions “exit,” “exit orifice,” “delivery orifice” or “drug delivery orifice,” and other similar expressions, as may be used herein include a member selected from the group consisting of a passageway; an aperture; an orifice; and a bore. The expression also includes an orifice that is formed or formable from a substance or polymer that erodes, dissolves or is leached from the outer wall to thereby form an exit orifice.

A drug “release rate” refers to the quantity of drug released from a dosage form per unit time, e.g., milligrams of drug released per hour (mg/hr). Drug release rates for drug dosage forms are typically measured as an in vitro rate of drug release, i.e., a quantity of drug released from the dosage form per unit time measured under appropriate conditions and in a suitable fluid. The dissolution tests utilized in the Examples described herein were performed on dosage forms placed in metal coil or metal cage sample holders attached to a USP Type VII bath indexer in a constant temperature water bath at 37° C. Aliquots of the release rate solutions were injected into a chromatographic system to quantify the amounts of drug released during the testing intervals.

By “release rate assay” is meant a standardized assay for the determination of the release rate of a compound from the dosage form tested using a USP Type VII interval release apparatus. It is understood that reagents of equivalent grade may be substituted in the assay in accordance with generally accepted procedures.

As used herein, unless otherwise specified, a drug release rate obtained at a specified time “following administration” refers to the in vitro drug release rate obtained at the specified time following implementation of an appropriate dissolution test. The time at which a specified percentage of the drug within a dosage form has been released may be referenced as the “Tₓ” value, where “x” is the percent of drug that has been released. For example, a commonly used reference measurement for evaluating drug release from dosage forms is the time at which 70% of drug within the dosage form has been released. This measurement is referred to as the “T₇₀” for the dosage form.

An “immediate-release dosage form” refers to a dosage form that releases drug substantially completely within a short time period following administration, i.e., generally within a few minutes to about 1 hour.

By “sustained release dosage form” is meant a dosage form that releases drug substantially continuously for many hours. Sustained release dosage forms in accord with the present invention exhibit T₇₀ values of at least about 8 to 20 hours and preferably 15 to 18 hours and more preferably about 17 hours or more. The dosage forms continuously release drug for sustained periods of at least about 8 hours, preferably 12 hours or more and, more preferably, 16-20 hours or more.

Dosage forms in accord with the present invention exhibit controlled release rates of a therapeutic agent for a prolonged period of time within the sustained release time period.

By “uniform release rate” is meant an average hourly release rate from the core that varies positively or negatively by no more than about 30% and preferably no more than about 25% and most preferably no more than 10% from either the preceding or the subsequent average hourly release rate as determined in a USP Type VII Interval Release Apparatus where the cumulative release is between about 25% to about 75%.

By “prolonged period of time” is meant a continuous period of time of at least about 4 hours, preferably 6-8 hours or more and, more preferably, 10 hours or more. For example, the exemplary osmotic dosage forms described herein generally begin releasing therapeutic agent at a uniform release rate within about 2 to about 6 hours following administration and the uniform rate of release, as defined above, continues for a prolonged period of time from about 25% to until at least about 75% and preferably at least about 85% of the drug is released from the dosage form. Release of therapeutic agent continues thereafter for several more hours although the rate of release is generally slowed somewhat from the uniform release rate.

By “C” is meant the concentration of drug in the blood plasma of a subject, generally expressed as mass per unit volume, typically nanograms per milliliter. For convenience, this concentration may be referred to as “plasma drug concentration” or “plasma concentration” herein which is intended to be inclusive of drug concentration measured in any appropriate body fluid or tissue. The plasma drug concentration at any time in units of hours following drug administration is referenced as Cₜᵢₘₑ, as in C₉ₕ or C₂₄ₕ, etc.

By “steady state” is meant the condition in which the amount of drug present in the blood plasma of a subject does not vary significantly over a prolonged period of time. A pattern of drug accumulation following continuous administration of a constant dose and dosage form at constant dosing intervals eventually achieves a “steady-state” where the plasma concentration peaks and plasma concentration troughs are essentially identical within each dosing interval. As used herein, the steady-state maximal (peak) plasma drug concentration is referenced as Cₘₐₓ and the minimal (trough) plasma drug concentration is referenced as Cₘᵢₙ. The times following drug administration at which the steady-state peak plasma and trough drug concentrations occur are referenced as the Tₘₐₓ and the Tₘᵢₙ, respectively.

Persons of skill in the art appreciate that plasma drug concentrations obtained in individual subjects will vary due to interpatient variability in the many parameters affecting drug absorption, distribution, metabolism and excretion. For this reason, unless otherwise indicated, mean values obtained from groups of subjects are used herein for purposes of comparing plasma drug concentration data and for analyzing relationships between in vitro dosage form dissolution rates and in vivo plasma drug concentrations.

By “high dosage” is meant drug loading efficiency of therapeutic agent within the dosage form that comprises 20% or more, preferably 40% or more, by weight of the drug layer composition tablet core of the dosage form.

It has been surprisingly discovered that sustained release dosage forms incorporating drug core compositions of high doses of low solubility therapeutic agent exhibiting T₇₀ values of about 10 to 20 hours and preferably 15 to 18 hours and more preferably at about 17 hours or more which release at a uniform release rate for a prolonged period of time can be prepared. Administration of such dosage forms once daily can provide therapeutically effective average steady-state plasma concentrations.

The exemplary sustained release dosage forms incorporating the drug core composition of the present invention, methods of preparing such dosage forms and methods of using such dosage forms described herein are directed to osmotic dosage forms for oral administration. In addition to osmotic systems as described herein, however, there are many other approaches to achieving sustained release of drugs from oral dosage forms known in the art. These different approaches may include, for example, diffusion systems such as reservoir devices and matrix devices, dissolution systems such as encapsulated dissolution systems (including, for example, “tiny time pills”) and matrix dissolution systems, combination diffusion/dissolution systems and ion-exchange resin systems as described in Remington's Pharmaceutical Sciences, 1990 ed., pp. 1682-1685. Therapeutic agent dosage forms that operate in accord with these other approaches are encompassed by the scope of the claims below to the extent that the drug release characteristics as recited in the claims describe those dosage forms either literally or equivalently.

Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. A significant advantage to osmotic systems is that operation is pH-independent and thus continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated in its entirety by reference herein. In particular, the following U.S. Patents, owned by the assignee of the present application, ALZA Corporation, directed to osmotic dosage forms, are each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

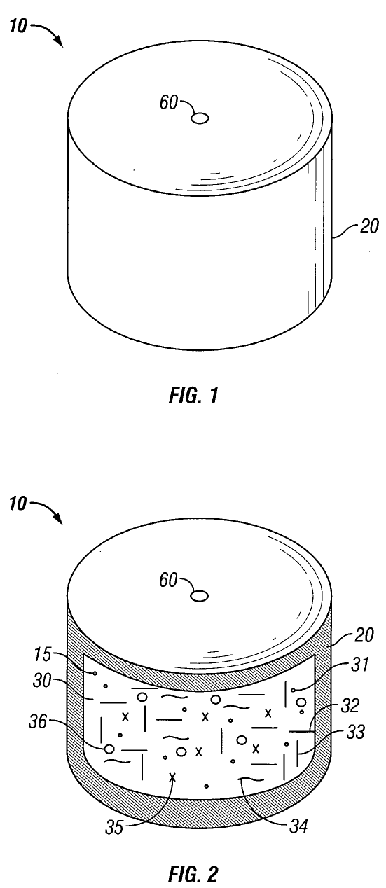

FIG. 1 is a perspective view of one embodiment of a sustained release osmotic dosage form in accord with the present invention. Dosage form 10 comprises wall 20 that surrounds and encloses an internal compartment (not seen in FIG. 1). The internal compartment contains a drug core composition comprising a therapeutic agent, or a pharmaceutically acceptable acid addition salt thereof, as described in more detail below. Wall 20 is provided with at least one drug delivery exit 60 for connecting the internal compartment with the exterior environment of use. Accordingly, following oral ingestion of dosage form 10, fluid is imbibed through wall 20 and the therapeutic agent is released through exit 60.

While the preferred geometrical embodiment in FIG. 1 illustrates a standard biconvex round shaped tablet, the geometry may embrace a capsule shaped caplet, oval, triangular, and other shapes designed for oral administration, including buccal, or sublingual dosage forms.

FIG. 2 is a cutaway view of FIG. 1 showing an embodiment of the present invention with internal compartment 15 containing a single component layer referred to herein as drug layer 30, comprising therapeutic agent drug 31 in an admixture with selected excipients adapted to increase solubility of drug layer 30 and provide an osmotic activity gradient for driving fluid from an external environment through wall 20 for forming a deliverable therapeutic agent formulation upon imbibition of fluid. As described in more detail below, the excipients include a suitable structural polymer referred to herein as drug carrier 32, represented by horizontal dashed lines and a suitable solubilizing agent referred to herein as surfactant 33 and is represented by vertical dashes.

Drug layer 30 excipients may further include a suitable lubricant 34 and an osmotically active agent, osmoagent 35, as represented by “x” symbols and a suitable binder 36.

In operation, following oral ingestion of dosage form 10, the osmotic activity gradient across wall 20 causes aqueous fluid of the gastrointestinal tract to be imbibed through the wall 20, thereby forming a deliverable therapeutic drug formulation, i.e., a solution or suspension, within the internal compartment. The deliverable drug formulation is released through exit 60 as fluid continues to enter the internal compartment. As release of drug formulation occurs, fluid continues to be imbibed thereby driving continued release. In this manner, drug is released in a sustained and continuous manner over an extended time period.

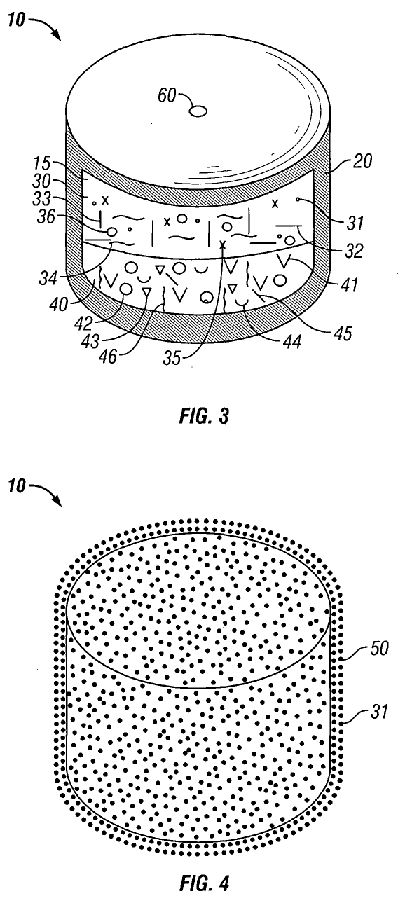

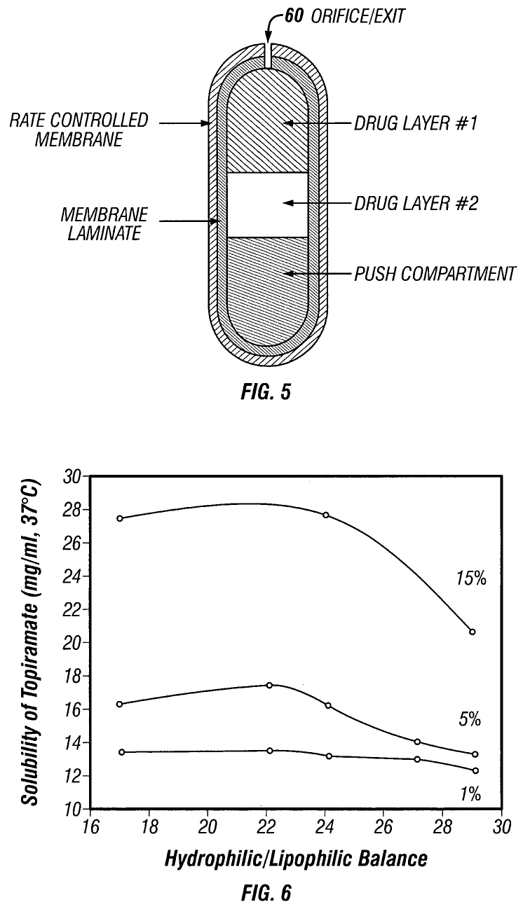

FIG. 3 is a cutaway view of FIG. 1 with an alternate embodiment of internal compartment 15 having a bilayer configuration. In this embodiment, internal compartment 15 contains a bilayered-compressed core having a first component drug layer 30 and a second component push layer 40. Drug layer 30, as described above with reference to FIG. 1, comprises therapeutic agent in an admixture with selected excipients.

As described in more detail below, second component push layer 40 comprises osmotically active component(s), but does not contain any active therapeutic agent. The components in push layer 40 typically comprise an osmoagent 42 and one or more osmopolymer 41, having relatively large molecular weights which exhibit swelling as fluid is imbibed. Additional excipients such as binder 43, lubricant 44, antioxidant 45 and colorant 46 may also be included in push layer 40. The second component layer 40 is referred to herein as an expandable or a push layer since, as fluid is imbibed, the osmopolymer(s) swell and push against the deliverable drug formulation of the first component drug layer to thereby facilitate release of the drug formulation from the dosage form.

In operation, following oral ingestion of the dosage form 10 as shown in FIG. 3, the osmotic activity gradient across wall 20 causes aqueous fluid to be imbibed through wall 20 thereby forming drug layer 30 into a deliverable formulation and concurrently swelling the osmopolymer(s) in push layer 40. The deliverable drug layer 30 is released through exit 60 as fluid continues to enter internal compartment 15 and push layer 40 continues to swell. As release of drug layer 30 occurs, fluid continues to be imbibed and the push layer continues to swell thereby driving continued release. In this manner, therapeutic agent is released in a sustained and continuous manner over an extended time period.

Drug layer 30, as described with reference to FIGS. 2 and 3, comprises a therapeutic agent in an admixture with selected excipients. Push layer 40, as described with reference to FIG. 3, comprises osmotically active component(s) but does not contain any therapeutic agent.

Drug layer 30 of the present invention comprises a drug core composition formed of three components: a pharmaceutically effective amount of therapeutic agent drug 31, or a pharmaceutically acceptable salt thereof, a carrier 32, and a solubilizing surfactant 33.

The lowly soluble therapeutic agent drug may include a member selected from the group consisting of acenocoumarol, acetaminophen, acetazolaminde, acetophenazine, acyclovir, albuterol, allopurinol, aprazolam, alteplase, amantidine, aminopyrine, amiloride, amiodarone, amitriptyline, amlodipine, amoxapine, amoxicillin, amphotericin B, ampicillin, apomorphine, aspirin, astemizole, atenolol, atracurium, atropine, auranofin, azathioprine, aztreonam, bacitracin, baclofen, beclomethasone, benazepril, bendroflumethiazide, betamethasone, biperiden, bitolterol, bromocriptine, buclizine, bumetanide, buprenorphine, busulfan, butorphanol, cadralazine, calcitriol, carbamazepine, carbidopa, carboplatin, cefaclor, cefazolin, cefoxitin, ceftazidime, cephalexin, chloramphenicol, chlordiazepoxide, chlorpheniramine, chlorpromazine, chlorpropamide, chlorthalidone, chlorzoxazone, cholestyramine, cimetidine, ciprofloxacin, cisapride, cisplatin, clarithromycin, clemastine, clonazepam, clotrimazole, clozapine, codeine, cyclizine, cyclobarbital, cyclosporine, cytarabine, chlorothiazide, cyclophosphamide, dacarbazine, deflazacort, deserpidine, desanoside, desogestrel, desoximetasone, dexamethasone, dextromethorphan, dezocine, diazepam, diclofenac, dicyclomine, diflunisal, digitoxin, digoxin, dihydroergotamine, dimenhydrinate, diphenoxylate, dipyridamole, disopyramide, dobutamine, domperidone, dopexamine, doxazosin, doxorubicin, doxycycline, droperidol, enalapril, enoximone, ephedrine, epinephrine, ergotoloids, ergovine, erythromycin, estazolam, estradiol,ethinyl estradiol,etodolac, etoposide, famotidine, felodipine, fenfluramine, fenoprofen, fentanyl, filgrastim, finasteride, fluconazole, fludrocortisone, flumazenil, flunisolide, fluocinonide, fluorourcil, fluoxetine, fluoxymesterone, fluphenazine, fluphenazine, flurbiprofen, flutamide, fluticasone, furosemide, ganciclovir, gemfibrizil, glipizide, glyburide, gramicidin, granisetron, guaifenesin, guanabenz, guanadrel, guanfacine, haloperidol, heparin, homatropine, hydralazine, hydrochlorothiazide, hydrocodone, hydrocortisone, hydromorphone, hydroxyzine, hyoscyamine, ibudilast, ibuprofen, isosorbide dinitrate, pseudoephedrine, cholchicine, secoverine, progesterone, naloxone, imipramine, indapamide, indomethacin, insulin, ipratropium, isocarboxazid, isopropamide, isosorbide,isotretinoin, isradipine, itraconazole, ketoconazole, ketoprofen, levonorgestrel, levorphanol, lidocaine, lindane, liothyronine, lisinopril, lithium, lomefloxacin, loperamide, loratadine, lorazepam, lovastatin, loxapine, mabuterol, maprotiline, mazindol, meclizine, medroxyprogesteron, mefenamic acid, melatonin, meperidine, mephentermine, mesalazine, mestranol, methdilazine, methotrimeprazine, methotrexate, methoxsalen, methoxypsoralen, methyclothiazide, methylphenidate, methylprednisolone, methyltestosterone, methysergide, metocurine iodide,metolazone, metronidazole, miconazole, midazolam, milrinone, minocycline, minoxidil, mitomycin, molsidomine, mometasone, morphine, mupirocin, muroctasin, nabumetone, nadolol, naltrexone, neostigmine, nicardipine, nicorandil, nicotine, nifedipine, nimodipine, nitrendipine, nitrofurantoin, nitroglycerin, norfloxacin, nystatin, octreotide, ofloxacin, omeprazole, oxaprozin, oxazepam, oxycodone, oxyphencyclimine, oxytetracycline, paclitaxel, paramethasone, paroxetine, pemoline, penicillin, pentaerythritol, pentamidine, pentazocine, pergolide, perphenazine, phenazopyridine, phenelzine, phenobarbitol, phenoxybenzamine, phenytoin, physostigmine, pimozide, pindolol, polythizide, prazepam, prazosin, prednisolone, prednisone, probucol, prochloperazine, procyclidine, propofol, propranolol, propylthiouracil, pyrimethamine, quinidine, ramipril, rescinnamine, reserpine, rifabutin, rifapentine, respiridone, salmeterol, sertraline, siagoside, simvastatin, spironolactone, sucralfate, sulfadiazine, sulfamethoxazole, sulfamethizole, sulindac, sulpiride, tamoxifen, tandospirone, temazepam, terazosin, terbinafine, terconazole, terfenadine, tetracaine, tetracycline, theophylline, thiethylperazine, thioridazine, thiothixene, thyroxine, timolol, topiramate, tranylcypromine, trazodone, tretinoin, triamcinolone, trimethoprim, triazolam, trichlormethiazide, trihexphenidyl, trioxsalen, tubocurarine, valproic acid, verapamil, vinblastine, vitamin B, warfarin, zidovudine, and lowly soluble derivatives, pro-drugs, isomers, and salts of the above. The doses these drugs that can be incorporated into the dosage form of the present invention can range from 1 microgram or less to about 750 milligrams, with an especially preferred range of 10 mg to 250 mg.

These drugs exhibit low solubility of less than 100 mg/ml with those most preferred for the present invention exhibiting solubility of less than 50 mg/ml.

The therapeutic salts are represented by a member selected from the group consisting of the following: anion salts such as acetate, adipate, benzenesulfonate, benzoate, bicarbonate, bitartrate, bromide, calcium edetate, camsylate, carbonate, chloride, citrate, dihydrochloride, edetate, edisylate, estolate, fumerate, gluceptate, gluconate, glutamate, glycollylarsanilate, hexylreorinate, hydrabamine, hydrobromide, hydrochloride, hydroxynaphthoate, iodide, isethionate, lactate, lactobionate, malate, maleate, mandelate, mesylate, methylbromide, methylnitrate, mucate, napsylate, nitrate, pamoate, pantothenate, phosphate, diphosphate, polygalacturonate, salicylate, stearate, subacetate, succinate, sulfate, tannate, tartrate, teoclate, triethiodide, or cation salts such as benzathine, chloroprocaine, choline, diethanolamine, ethylenediamine, meglumine, procaine, aluminium, calcium, lithium, magnesium, potassium, sodium, zinc, polymer/drug complexes such as cyclodextrinates, polyvinylpyrrolidonates, and the like.

When drug 31 is present in high dosage amounts, greater than 20% of the drug layer 30 by weight, the present invention provides a beneficial increased solubility of the lowly soluble drug to provide for creation of a deliverable drug layer 30. Additionally, the present invention provides a potentially beneficial increased bioavailability of the lowly soluble drug by increasing its solubility and wetted surface for greater bioadhesion to the gastrointestinal tract mucosa. The wetting properties of solubilizing surfactants can also have the effect of preventing the released drug and hydrogel carrier from agglomerating, thereby leading to a more complete spreading of the dispensed drug composition onto the absorbable surfaces of the gastrointestinal tract which increased surface area provides more absorption surface area to increase the rate and extent of drug absorbed and increase the therapeutic response. Moreover, the solubilizing surfactant can impart adhesive character to the dispensed drug/hydrogel which adhesive character can prolong in time the contact that the drug/hydrogel makes with the absorbable mucosal tissue of the gastrointestinal tract giving more time for the drug to be spread onto and absorbed once delivered. In yet another potential beneficial effect, the solubilizing surfactant can additionally increase the permeability of mucosal membranes to the drug molecule which permeability enhancement can lead also to enhanced bioavailability of the drug and enhanced therapeutic response.

When drug 31 of the present invention is present in low dosage amounts, less than 20% of drug layer 30, the present invention provides a beneficial increased bioavailability of the lowly soluble drug by increasing its solubility and wetted surface for greater bioadhesion to the gastrointestinal tract mucosa and enhanced permeability of the mucosal surfaces. The increased drug solubility, the increased surface contact area on the mucosal tissue, the increased contact time to the mucosal tissue, and permeability enhancement of the mucosal tissue to the drug molecule can individually or compositely contribute to the overall therapeutic enhancement of the drug by the present invention.

Drug 31 is exemplified herein through the use of topiramate and phenytoin, each of which is lowly soluble and therapeutically required to be delivered in high doses. Both drugs are in the therapeutic category of anti-convulsants although the drugs may be therapeutic for other indications as well. Solubility of neat topiramate was measured in de-ionized water at 37 degrees centigrade to be 13 mg/ ml. The recommended therapy of the topiramate involves dosing initially at 25-50 mg/day followed by titration in weekly increments of 25-50 mg upward to an effective dose. Typical effective dose can be up to 400 mg per day.

The solubility of phenytoin is 0.02 mg/ml as reported in Analytical Profiles of Drug Substances Volume 13, Edited by Klaus Florey (Academic Press, New York, 1984) p 425. The recommended therapy for phenytoin is 100 mg doses three to four times per day. The recommended doses and dosing regimens of each drug are described in Physician's Desk Reference 56^{th} Edition (Medical Economics Company, New Jersey, 2002) p. 2595 and 2626.

Structural polymer carrier 32 comprises a hydrophilic polymer which provides cohesiveness to the blend so durable tablets can be made. The structural polymer also provides during the operation of the delivery system of the present invention a hydrogel with viscosity. This viscosity suspends drug particles to promote partial or complete dissolution of the drug prior to delivery from the dosage form.

If the present invention is used in an erodible matrix application, the molecular weight of the structural polymer is selected to modify the erosion rate of the system. High molecular weight polymers are used to produce slow erosion rate and slow delivery of drug, low molecular weight polymers produce faster erosion rate and faster release of drug. A blend of high and low molecular weight structural polymers produces an intermediate delivery rate.

If the present invention is used in a nonerodible porous matrix, the molecular weight of the structural polymer is selected to provide a hydrogel with viscosity within the pores of the matrix. This viscosity suspends drug particles to promote partial or complete dissolution of the drug in the presence of the solubilizing surfactant prior to delivery from the pores of the dosage form.

Carrier 32 provides a hydrophilic polymer particle in the drug composition that contributes to the controlled delivery of active agent. Representative examples of these polymers are poly(alkylene oxide) of 50,000 to 8 million and more preferably of 100,000 to 750,000 number-average molecular weight, including poly(ethylene oxide), poly(methylene oxide), poly(butylene oxide) and poly(hexylene oxide); and a poly(carboxymethylcellulose) of 40,000 to 1,000,000 400,000 number-average molecular weight, represented by poly(alkali carboxymethylcellulose), poly(sodium carboxymethylcellulose), poly(potassium carboxymethylcellulose) poly(calcium carboxymethylcellulose), and poly(lithium carboxymethylcellulose). The drug composition can comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among those polymers are the poly(ethylene oxide) of 100,000-300,000 number average molecular weight. Carriers that erode in the gastric environment, i.e., bioerodible carriers, are especially preferred.

Other carriers that may be incorporated into drug layer 30 include carbohydrates that exhibit sufficient osmotic activity to be used alone or with other osmoagents. Such carbohydrates comprise monosaccharides, disaccharides and polysaccharides. Representative examples include maltodextrins (i.e., glucose polymers produced by the hydrolysis of grain starch such as rice or corn starch) and the sugars comprising lactose, glucose, raffinose, sucrose, mannitol, sorbitol, zylitol and the like. Preferred maltodextrins are those having a dextrose equivalence (DE) of 20 or less, preferably with a DE ranging from about 4 to about 20, and often 9-20. Maltodextrin having a DE of 9-12 and molecular weight of about 1,600 to 2,500 has been found most useful.

Carbohydrates described above, preferably the maltodextrins, may be used in the drug layer 30 without the addition of an osmoagent, and obtain the desired release of therapeutic agent from the dosage form, while providing a therapeutic effect over a prolonged period of time and up to 24 hours with once-a-day dosing.

The presently preferred range of concentration of structural polymer within the present invention for osmotic delivery systems is 5 to 50 weight percent of polyoxyethylene 200,00 molecular weight (Polyox® N80), with an especially preferred range of 5-15 weight percent.

Drug layer 30 further comprises a therapeutically acceptable solubilizing agent, surfactant 33 represented by vertical dashes in FIG. 2 and FIG. 3. Acceptable solubilizing agents include, for example, a surfactant of polyoxyl 40 stearate and polyoxyl 50 stearate can be used as the solubilizing surfactant. Yet another class of surfactant useful in forming the dissolved drug is triblock co-polymers of ethylene oxide/propylene oxide/ethylene oxide, also known as poloxamers. In this class of surfactants, the hydrophilic ethylene oxide ends of the surfactant molecule and the hydrophobic midblock of propylene oxide of the surfactant molecule serve to dissolve and suspend the drug in the pumpable hydrogel. Other surfactants that are solids at room temperature include members selected from the group essentially consisting of sorbitan monopalmitate, sorbitan monostearate, glycerol monostearate and polyoxyethlene stearate (self emulsifying), polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether with butylated hydroxyanisole and citric acid added as preservatives, polyoxyethylene 2 cetyl ether with butylated hydroxyanisole and citric acid added as preservatives, polyoxyethylene 10 cetyl ether with butylated hydroxyanisole and citric acid added as preservatives, polyoxyethylene 20 cetyl ether with butylated hydroxyanisole and citric acid added as preservatives, polyoxyethylene 2 stearyl ether with butylated hydroxyanisole and citric acid added as preservatives, polyoxyethylene 10 stearyl ether with butylated hydroxyanisole and citric acid added as preservatives, polyoxyethylene 20 stearyl ether with butylated hydroxyanisole and citric acid added as preservatives, polyoxyethylene 21 stearyl ether with butylated hydroxyanisole and citric acid added as preservatives, polyoxyethylene 20 oleyl ether with butylated hydroxyanisole and citric acid added as preservatives, polyoxyethylene 40 stearate, polyoxyethylene 50 stearate, polyoxyethylene 100 stearate, sorbitan monopalmitate, sorbitan monostearate, sorbitan tristearate, polyoxyethylene 4 sorbitan monostearate, polyoxyethylene 20 sorbitan tristearate, and the like. An especially preferred family of surfactants are a:b:a triblock co-polymers of ethylene oxide:propylene oxide:ethylene oxide. The “a” and “b” represent the average number of monomer units for each block of the polymer chain. These surfactants are commercially available from BASF Corporation of Mount Olive, N.J., in a variety of different molecular weights and with different values of “a” and “b” blocks. For example, Lutrol® F127 has a molecular weight range of 9,840 to 14,600 and where “a” is approximately 101 and “b” is approximately 56, Lutrol F87 represents a molecular weight of 6,840 to 8,830 where “a” is 64 and “b” is 37, Lutrol F108 represents an average molecular weight of 12,700 to 17,400 where “a” is 141 and “b” is 44, and Lutrol F68 represents an average molecular weight of 7,680 to 9,510 where “a” has a value of about 80 and “b” has a value of about 27. A resource of surfactants including solid surfactants and their properties is available in McCutcheon's Detergents and Emulsifiers, International Edition 1979 and McCutcheon's Detergents and Emulsifiers, North American Edition 1979. Other sources of information on properties of solid surfactants include BASF Technical Bulletin Pluronic & Tetronic Surfactants 1999 and General Characteristics of Surfactants from ICI Americas Bulletin 0-1 10/80 5M.

One of the characteristics of surfactants tabulated in these references is the HLB value, or hydrophilic lipophilic balance value. This value represents the relative hydroplicility and relative hydrophobicity of a surfactant molecule. Generally, the higher the HLB value, the greater the hydrophilicity of the surfactant while the lower the HLB value, the greater the hydrophobicity. For the Lutrol® molecules, for example, the ethylene oxide fraction represents the hydrophilic moiety and the propylene oxide fraction represents the hydrophobic fraction. The HLB values of Lutrol F127, F87, F108, and F68 are respectively 22.0, 24.0, 27.0, and 29.0.

Surfactants, typically have poor cohesive properties and therefore do not compress as hard, durable tablets. Furthermore, surfactants are in the physical form of liquid, pastes, or waxy solids at standard temperatures and conditions and are inappropriate for tabletted oral pharmaceutical dosage forms. The aforementioned surfactants have been surprisingly found to function in the present invention by enhancing the solubility and potential bioavailability of low solubility drugs delivered in high doses.

Surfactant 33 can be one surfactant or a blend of surfactants. The surfactants are selected such that they have values that promote the dissolution and solubility of the drug. A high HLB surfactant can be blended with a surfactant of low HLB to achieve a net HLB value that is between them, if a particular drug requires the intermediate HLB value. Surfactant 33 is selected depending upon the drug being delivered; such that the appropriate HLB grade is utilized.

The present invention involves a method to match the appropriate solid surfactant or blend of surfactants with a particular pharmaceutical active agent to produce the solubilizing core, or S-Core of the present invention. The method involves preparing aqueous solutions of surfactants spanning a range of HLB values and a range of concentrations. Then, pharmaceutical agent is added in excess to the surfactant solutions and the saturated solubility of the pharmaceutical active agent is then measured by an appropriate analytical method such as ultraviolet spectroscopy, chromatographic methods, or gravimetric analysis. Then, the solubility values are plotted as a function of HLB and as a function of surfactant concentration. The maximal point of solubility generated in the plots at the different concentrations reveals the solid surfactant or blend of surfactants for use in the S-Core of the present invention.

In those delivery systems that are constructed with more than one drug layer, a drug concentration gradient ratio between the two drug layers is defined to be in the range of 1.0 to 2.0. This ratio, when combined with application of surfactant at certain drug to surfactant ratio can be used to achieve an acceptable ascending release rate profile as targeted.

The ratio of drug to surfactant is defined to be in the range of about 0.5:1 to about 2.0:1 in both drug layers to achieve a functional release rate profile.

A variety of processing techniques can be used to promote uniformity of mixing between the drug and surfactant 33 in drug layer 30. In one method, the drug and surfactant are each micronized to a nominal particle size of less than about 200 microns. Standard micronization processes such as jet milling, cryogrinding, bead milling, and the like can be used. Alternately, the drug and surfactant can be dissolved in a common solvent to produce mixing at the molecular level and co-dried to a uniform mass. The resulting mass can be ground and sieved to a free-flowing powder. The resulting free-flowing powder can be granulated with wet mass sieving or fluid bed granulation with the structural polymer carrier to form the drug granulation of the present invention. Alternately, drug 31 and surfactant 33 can be melted together at elevated temperature to encapsulate the drug in surfactant, and then congealed to room temperature. The resulting solid can be ground, sized, and granulated with the structural polymer carrier.

In another manufacturing process, the drug and surfactant can be dissolved in a common solvent or blend of solvents and spray dried to form a co-precipitate that is incorporated with the structural polymer by standard granulation processing by fluid bed processing or wet mass sieving. In yet another manufacture, the drug and surfactant can be dissolved in a common solvent or blend of solvents which drug/surfactant solution is sprayed onto the structural polymer carrier directly in a fluid bed granulation process.

The amount of carrier 32 and surfactant 33 formulated within drug layer 30 must be appropriately selected and controlled. Excessive carrier 32 creates a hydrated drug layer that is too viscous to be delivered from the dosage form through exit 60 while too little carrier 32 does not afford sufficient functional viscosity to control delivery. Insufficient levels of structural carrier 32 also create manufacturing problems in that the tablet by not having sufficient structural integrity is unable to resist crumbling and degradation by abrasion or physical insult. Similarly, too much surfactant 33 creates structural instability of the tablet core while too little does not provide sufficient solubilizing of the drug layer 30 to allow it to form a deliverable solution or suspension. The amount of carrier 32 in drug layer 30 should be 1% to 80% and preferably 5% to 50% and more preferably 10% to 40%. The amount of surfactant 33 in the dosage form should be 5 to 50% and preferably 5% to 40%. Low doses will require amounts of carrier in the higher ranges whereas higher doses will require doses of carrier in the lower ranges.

Dosage form 30 may further comprise lubricant 34 represented by a horizontal wavy line in FIG. 2 and FIG. 3. The lubricant is used during tablet manufacture to prevent adherence to die walls or punch faces. Typical lubricants include magnesium stearate, sodium stearate, stearic acid, calcium stearate, magnesium oleate, oleic acid, potassium oleate, caprylic acid, sodium stearyl fumarate, and magnesium palmitate or blends of such lubricants. The amount of lubricant present in the therapeutic composition is 0.01 to 20 mg.

Drug layer 30 may further comprise a therapeutically acceptable vinyl polymer binder 36 represented by small circles in FIG. 2 and FIG. 3. The vinyl polymer comprises a 5,000 to 350,000 average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Dosage form 10 and the therapeutic composition comprises 0.01 to 25 mg of the binder or vinyl polymer that serves as a binder. Representative of other binders include acacia, starch and gelatin.

Drug layer 30 will be a dry composition formed by compression of the carrier, surfactant and drug core composition as one layer and the push composition as the other layer in contacting relation.

Drug layer 30 is formed as a mixture containing a therapeutic agent, carrier and the surfactant, that when contacted with biological fluids in the environment of use provides a slurry, solution or suspension of the compound that may be dispensed by the action of the push layer. The drug layer may be formed from particles by comminution that produces the size of the drug and the size of the accompanying polymer used in the fabrication of the drug layer, typically as a core containing the compound, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

Drug layer 30 may further comprise disintegrants. Disintegrants may be selected from starches, clays, celluloses, algins and gums and crosslinked starches, celluloses and polymers. Representative disintegrants include corn starch, potato starch, croscarmelose, crospovidone, sodium starch glycolate, Veegum HV, methylcellulose, agar, bentonite, carboxymethylcellulose, alginic acid, guar gum, low-substituted hydroxypropyl cellulose, microcrystalline cellulose, and the like.

The therapeutic agent may be provided in the drug layer in amounts from 1 ug to 750 mg per dosage form, preferably 1 mg to 500 mg per dosage form, and more preferably 10 mg to 400 mg, depending upon the therapeutic agent and required dosing level that must be maintained over the delivery period, i.e., the time between consecutive administrations of the dosage forms. More typically, loading of compound in the dosage forms will provide doses of compound to the subject ranging from 20 mg to 350 mg and more usually 40 mg to 200 mg per day. Generally, if a total drug dose of more than 200 mg per day is required, multiple units of the dosage form may be necessarily administered at the same time to provide the required amount of drug.

As a representative compound of the compounds having therapeutic activity described herein, immediate release topiramate is typically administered for treatment of epilepsy at a starting dose of about 25 to 50 mg per day. This regimen continues over a period of a week. Then, the patient is titrated upward each week in increments of 25 to 50 mg per day depending upon tolerability until an effective dose is reached. The effective dose range for this indication has been determined to be generally about 400 mg/day.

As a representative compound of the compounds having therapeutic activity described herein, immediate release phenytoin is typically administered at a starting dose of about 100 mg, administered in two or four doses per day. The effective dose range has been determined to be generally 200 mg/day-400 mg/day. Observation of tolerability and need for additional clinical effect over the starting dose often results in the dose being increased up to a regimen of 200 mg three times per day.

Push layer 40 comprises a displacement composition in contacting layered arrangement with the first component drug layer 30 as illustrated in FIG. 3. Push layer 40 comprises osmopolymer 41 that imbibes an aqueous or biological fluid and swells to push the drug composition through the exit means of the device. A polymer having suitable imbibition properties may be referred to herein as an osmopolymer. The osmopolymers are swellable, hydrophilic polymers that interact with water and aqueous biological fluids and swell or expand to a high degree, typically exhibiting a 2-50 fold volume increase. The osmopolymer can be non-crosslinked or crosslinked.

Push layer 40 comprises 20 to 375 mg of osmopolymer 41, represented by “V” symbols in FIG. 3. Osmopolymer 41 in layer 40 possesses a higher molecular weight than osmopolymer 32 in drug layer 20.

Representatives of fluid-imbibing displacement polymers comprise members selected from poly(alkylene oxide) of 1 million to 15 million number-average molecular weight, as represented by poly(ethylene oxide), and poly(alkali carboxymethylcellulose) of 500,000 to 3,500,000 number-average molecular weight, wherein the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rite® polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

Push layer 40 comprises 0 to 75 mg, and presently 5 to 75 mg of an osmotically effective compound, osmoagent 42, represented by large circles in FIG. 3. The osmotically effective compounds are known also as osmoagents and as osmotically effective solutes. Osmoagent 42 that may be found in the drug layer and the push layer in the dosage form are those that exhibit an osmotic activity gradient across the wall 20. Suitable osmoagents comprise a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid, raffinose, sucrose, glucose, lactose, sorbitol, inorganic salts, organic salts and carbohydrates.

Push layer 40 may further comprises a therapeutically acceptable vinyl polymer 43 represented by triangles in FIG. 3. The vinyl polymer comprises a 5,000 to 350,000 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Push layer contains 0.01 to 25 mg of vinyl polymer.

Push layer 40 may further comprise 0 to 5 mg of a nontoxic colorant or dye 46, identified by vertical wavy lines in FIG. 3. Colorant 35 includes Food and Drug Administration Colorant (FD&C), such as FD&C No. 1 blue dye, FD&C No. 4 red dye, red ferric oxide, yellow ferric oxide, titanium dioxide, carbon black, and indigo.

Push layer 40 may further comprise lubricant 44, identified by half circles in FIG. 3. Typical lubricants comprise a member selected from the group consisting of sodium stearate, potassium stearate, magnesium stearate, stearic acid, calcium stearate, sodium oleate, calcium palmitate, sodium laurate, sodium ricinoleate and potassium linoleate, and blends of such lubricants. The amount of lubricant included in the push layer 40 is 0.01 to 10 mg.

Push layer 40 may further comprise an antioxidant 45, represented by slanted dashes in FIG. 3 to inhibit the oxidation of ingredients comprising expandable formulation 40. Push layer 40 comprises 0.00 to 5 mg of an antioxidant. Representative antioxidants comprise a member selected from the group consisting of ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiary butylphenol, alpha-tocopherol, and propylgallate.

FIG. 4 depicts a preferred embodiment of the present invention comprising an overcoat 50 of drug 31 on the dosage form of FIG. 3. Dosage form 10 of FIG. 4 comprises an overcoat 50 on the outer surface of wall 20 of dosage form 10. Overcoat 50 is a therapeutic composition comprising 1 ug to 200 mg of drug 31 and 5 to 200 mg of a pharmaceutically acceptable carrier selected from the group consisting of alkylcellulose, hydroxyalkylcellulose and hydroxypropylalkylcellulose. The overcoat is represented by methylcellulose, hydroxyethylcellulose, hydroxybutylcellulose, hydroxypropylcellulose, hydroxypropylmethylcellulose, hydroxypropylethylcellulose and hydroxypropylbutylcellulose, polyvinyl pyrrolidone/vinyl acetate copolymer, polyvinyl alcohol-polyethylene graft copolymer, and the like. Overcoat 50 provides therapy immediately as overcoat 50 dissolves or undergoes dissolution in the presence of gastrointestinal fluid and concurrently therewith delivers drug 31 into the gastrointestinal tract for immediate therapy. Drug 31 in overcoat 50 can be the same or different than the drug 31 in drug layer 30.

Exemplary solvents suitable for manufacturing the dosage form components comprise aqueous or inert organic solvents that do not adversely harm the materials used in the system. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Wall 20 is formed to be permeable to the passage of an external fluid, such as water and biological fluids, and it is substantially impermeable to the passage of drug 31, osmagent, osmopolymer and the like. As such, it is semipermeable. The selectively semipermeable compositions used for forming the wall are essentially nonerodible and they are substantially insoluble in biological fluids during the life of the dosage form.

Representative polymers for forming wall 20 comprise semipermeable homopolymers, semipermeable copolymers, and the like. Such materials comprise cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like, wherein the organic moieties contain from one to twelve carbon atoms, and preferably from one to eight carbon atoms.

The semipermeable compositions typically include a member selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanoate and cellulose tripropionate; cellulose diesters having a DS of 2.2 to 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semipermeable polymers are known in U.S. Pat. No. 4,077,407, and they can be synthesized by procedures described in Encyclopedia of Polymer Science and Technology, Vol. 3, pp. 325-354 (1964), Interscience Publishers Inc., New York, N.Y.

Additional semipermeable polymers for forming the outer wall 20 comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3,276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodium styrenesulfonate); semipermeable poly(vinylbenzyltrimethylammonium chloride); and semipermeable polymers exhibiting a fluid permeability of 10⁻⁵ to 10⁻⁵ (cc. mil/cm hr.atm), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff (1971) CRC Press, Cleveland, Ohio. Wall 20 can optionally be formed as two or more lamina such as described in U.S. Pat. No. 6,210,712.

Wall 20 may also comprise a flux-regulating agent. The flux regulating agent is a compound added to assist in regulating the fluid permeability or flux through wall 20. The flux-regulating agent can be a flux-enhancing agent or a flux-decreasing agent. The agent can be preselected to increase or decrease the liquid flux. Agents that produce a marked increase in permeability to fluid such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents may include polyhydric alcohols, polyalkylene glycols, polyalkylenediols, polyesters of alkylene glycols, and the like. Typical flux enhancers include polyethylene glycol 300, 400, 600, 1500, 4000, 6000 and the like; low molecular weight glycols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Presently preferred flux enhancers include the group of difunctional block-copolymer polyoxyalkylene derivatives of propylene glycol known as Lutrols. Representative flux-decreasing agents include phthalates substituted with an alkyl or alkoxy or with both an alkyl and alkoxy group such as diethyl phthalate, dimethoxyethyl phthalate, dimethyl phthalate, and [di(2-ethylhexyl) phthalate], aryl phthalates such as triphenyl phthalate, and butyl benzyl phthalate; polyvinyl acetates, triethyl citrate, Eudragit; insoluble salts such as calcium sulfate, barium sulfate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based wall forming materials, and the like.

Other materials may be included in the semipermeable wall material for imparting flexibility and elongation properties, to make wall 20 less brittle and to render tear strength. Suitable materials include phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

Pan coating may be conveniently used to provide the walls of the completed dosage form. In the pan coating system, the wall-forming composition for wall 20 is deposited by successive spraying of the appropriate wall composition onto the compressed single or bilayered core comprising the drug layer for the single layer core or the drug layer and the push layer for the laminated core, accompanied by tumbling in a rotating pan. A pan coater is used because of its availability at commercial scale. Other techniques can be used for coating the compressed core. Once coated, the wall is dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the wall or walls of the dosage form may be formed in one technique using the air-suspension procedure. This procedure consists of suspending and tumbling the compressed single or bilayer core in a current of warmed air and the semipermeable wall forming composition, until the wall is applied to the core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent for the wall forming material. An Aeromatic® air-suspension coater can be used employing a cosolvent.

Dosage forms in accord with the present invention are manufactured by standard techniques. For example, the dosage form may be manufactured by the wet granulation technique. In the wet granulation technique, the drug, carrier and surfactant are blended using an organic solvent, such as denatured anhydrous ethanol, as the granulation fluid. The remaining ingredients can be dissolved in a portion of the granulation fluid, such as the solvent described above, and this latter prepared solution is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for up to 10 minutes. The composition is pressed into a layer, for example, in a Manesty® press or a Korsch LCT press. For a bilayered core, the drug-containing layer is pressed and a similarly prepared wet blend of the push layer composition, if included, is pressed against the drug-containing layer. The intermediate compression typically takes place under a force of about 50-100 newtons. Final stage compression typically takes place at a force of 3500 newtons or greater, often 3500-5000 newtons. The single or bilayer compressed cores are fed to a dry coater press, e.g., Kilian® Dry Coater press, and subsequently coated with the wall materials as described above. A like procedure is employed for those cores that are manufactured with a push layer and more than one drug layer, typically on a Korsch multi-layer press.

One or more exit orifices are drilled in the drug layer end of the dosage form, and optional water soluble overcoats, which may be colored (e.g., Opadry colored coatings) or clear (e.g., Opadry Clear), may be coated on the dosage form to provide the finished dosage form.

In another manufacture the drug and other ingredients comprising the drug layer are blended and pressed into a solid layer. The layer possesses dimensions that correspond to the internal dimensions of the area the layer is to occupy in the dosage form, and it also possesses dimensions corresponding to the second push layer, if included, for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, if included, a layer of osmopolymer composition is placed in contact with the layer of drug in a like manner. The layering of the drug formulation and the osmopolymer layer can be fabricated by conventional two-layer press techniques. The compressed cores then may be coated with the semipermeable wall material as described above.

Another manufacturing process that can be used comprises blending the powdered ingredients for each layer in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

Exit 60 is provided in each dosage form. Exit 60 cooperates with the compressed core for the uniform release of drug from the dosage form. The exit can be provided during the manufacture of the dosage form or during drug delivery by the dosage form in a fluid environment of use.

Exit 60 may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semipermeable wall; a gelatinous filament; a water-removable poly(vinyl alcohol); a leachable compound, such as a fluid removable pore-former selected from the group consisting of inorganic and organic salt, oxide and carbohydrate.

The exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

The exit can have any shape, such as round, triangular, square, elliptical and the like for the uniform metered dose release of a drug from the dosage form.

The dosage form can be constructed with one or more exits in spaced-apart relation or one or more surfaces of the dosage form.

Drilling, including mechanical and laser drilling, through the semipermeable wall can be used to form the exit orifice. Such exits and equipment for forming such exits are disclosed in U.S. Pat. Nos. 3,916,899, by Theeuwes and Higuchi and in U.S. Pat. No. 4,088,864, by Theeuwes, et al. It is presently preferred to utilize a single exit orifice.

The release from the present invention provides efficacious therapy over 24 hours. This dosage form releases drug 31 for about 16-24 hours after administration with an optional immediate release drug overcoat delivery and controlled drug delivery continuing thereafter until the core ceases to release drug.

Representative dosage forms had T₇₀ values of greater than 10 hours and released topiramate for a continuous period of time of more than about 16 hours. Within about 2 hours following administration, each of the different dosage forms were releasing topiramate from the core at a uniform zero order or uniform ascending rate, depending upon the composition of drug layer and push layers, that continued for a prolonged period of time of about 8 to 14 hours or more. Following the prolonged period of delivery drug continues to be delivered for several more hours until the dosage form is spent or expelled from the GI tract.

In a bilayer embodiment of once-a-day dosage forms in accord with the present invention, the dosage forms have a T₇₀ of about 15 to 18 hours and preferably about 17 hours and provided release of topiramate for a continuous period of time of at least about 24 hours. Within about 2 hours following administration, topiramate is being released at a release rate that continues for a prolonged period of time. Following this prolonged period of uniform release rates, drug release continues for several more hours until the dosage form is spent.

Dosage forms of this invention exhibit sustained release of drug over a continuous time period that includes a prolonged time when drug is released at a uniform release rate as determined in a standard release rate assay such as that described herein.

The method is practiced with dosage forms that are adapted to release the compound at various rates of release between about 1%/hr to about 12%/hr over a prolonged time period of at least about 12 hours, preferably 14 hours or more.

The practice of the foregoing methods by orally administering a dosage form to a subject once a day for therapeutic treatment is preferred.

Preferred methods of manufacturing dosage forms of the present invention are generally described in the examples below. All percentages are weight percent unless otherwise noted.

### DESCRIPTION OF EXAMPLES OF THE INVENTION

The following examples are illustrative of the present invention and they should not be considered as limiting the scope of the invention in any way, as these examples and other equivalents thereof will become apparent to those versed in the art in light of the present disclosure, drawings and accompanying claims.

### EXAMPLE 1

### Method of Practicing the Invention.

A drug layer of the present invention was prepared as follows.

Aqueous solutions of five surfactants were prepared. The selected surfactants were four grades of ethylene oxide/propylene oxide/ethylene oxide (Lutrol grades F127, F87, F 108, and F68) and PEG-40 stearate (Myrj 52). Solutions were made at concentrations of 1, 5, and 15 weight percent. The aqueous surfactant blends solutions were chilled as necessary to promote complete dissolution of the surfactant prior to drug solubility studies. Each surfactant had a different HLB value and spanned a range of 16.9 to 29 HLB units.

The aqueous surfactant solutions were equilibrated to constant temperature in a 37° C. water bath. Then, neat topiramate drug was added slowly with stirring in approximately 10 mg increments to the surfactant solutions until no more drug dissolved. A control sample of drug dissolved in de-ionized water without surfactant was included for comparison purposes. The resulting saturated solutions of drug were filtered through 0.8-micron filters and analyzed for drug concentration by refractive index chromatography. The resulting solubility values were plotted as a 30 function of both surfactant concentration and the hydrophilic-lipophilic balance value of each surfactant. FIG. 6 was constructed from the solubility values obtained and HLB data for each surfactant utilized.

This method reveals three insights. Referring to FIG. 6, topiramate solubility in water is increased by each surfactant. Drug solubility is higher in the presence of each surfactant compared to the control where the solubility in de-ionized water without surfactant was 13.0 mg/ml. Second, a high concentration of surfactant is more effective in solubilizing drug than a low concentration. Third, the HLB values most effective to increase solubility of this drug are at the lower end, in the range of 16.9 to 22. The three concentrations of surfactant each form the maximal solubility of topirate with an HLB encompassing this range of HLB values. Therefore, Lutrol F 127or Lutrol F127 blended with Myrj 52, which has an HLB value of 16.9, is preferred for topiramate in the present invention.

Following this finding, a drug core composition of the present invention was prepared. First, 55 grams of topiramate, 30 grams of granular Lutrol F 127, 11.5 grams of the polyethylene oxide (PEO) N80, and 3 grams of polyvinyl pyrrolidone (PVP) 2932 were passed through a #40 mesh sieve and the composition was dry mixed to a uniform blend wherein the PVP acts as a binder and the PEO acts as the carrier. The molecular weight of the polyethylene oxide was 200,000 grams per mole and the molecular weight of the polyvinyl pyrrolidone was approximately 10,000. The polyoxyethylene serves as carrier and structural polymer 32. The polyvinyl pyrrolidone serves as the drug layer binder 36. The dry mixture was then wetted with anhydrous ethyl alcohol SDA 3A anhydrous and stirred to form a uniformly wetted mass. The wet mass was then passed through a 20-mesh sieve, forming damp noodles. The noodles were air dried at ambient conditions overnight, then passed again through a #20 mesh sieve, forming free-flowing granules. Finally, 0.5 grams of drug layer lubricant 34 magnesium stearate was passed through a # 60 mesh sieve over the granules and tumble mixed into the granules. This formed the drug layer composition granulation.

An expandable composition granulation was prepared in a similar manner. First, 89 grams of polyethylene oxide 303, 7 grams of sodium chloride, and 3 grams of hydroxypropyl methylcellulose E5 were passed through a #40 mesh sieve and dry mixed. The polyethylene oxide had a molecular weight of approximately 7,000,000 and the hydroxypropyl methylcellulose had a molecular weight of approximately 11,300. The polyethylene oxide served as the push layer osmopolymer 41 and the hydroxypropyl methylcellulose provided the push layer binder 43. Next, the dry mixture was wetted with anhydrous ethyl alcohol SDA 3A and mixed to a uniform damp mass. The mass was passed through a #20 mesh sieve forming noodles that were air dried overnight. Next, the noodles were passed again through a #20 mesh sieve forming free-flowing granules. Finally, 0.5 grams of minus #60 mesh magnesium stearate, push layer lubricant 44, was tumbled into the blend. This formed the expandable composition granulation.

A portion of the drug core composition granulation weighing 182 mg was filled into a 3/16-inch diameter die cavity and lightly tamped with 3/16 inch biconvex round tablet tooling. Then, 60 mg of the expandable composition granulation was filled into the die and compressed and laminated to the drug layer using a force of 0.5 tons with a Carver press. Six of these bilayer tablets were compressed.

Next, the tablets were coated with three layers. First, a solution was prepared by dissolving 57 grams of hydroxyethyl cellulose 250L and 3 grams of polyethylene glycol in 940 grams of de-ionized water. The hydroxyethyl cellulose had a molecular weight of approximately 90,000 and the polyethylene glycol had a molecular weight of 3,350. This formed a smoothing coat solution to provide a smooth coatable surface for subsequent coatings.

The six active tablets mixed into a tablet bed of placebo tablets that weighed 0.5 kg. The tablet bed was coated with this smoothing coat solution in an Aeromatic coater. The solution was applied in a current of warm, dry air until approximately 4 mg of coating weight was accumulated on each active tablet. The coating solution was stirred continuously during the coating process. The resulting smoothing coat produced a smooth tablet substrate and rounded the corners of the tablets. This smoothing coat is optional and is especially useful to round the corners of the tablets where tablet lands have flash from the compression process. The resulting smooth tablets were dried in a 40° C. force air oven overnight.

The next coating solution was prepared by dissolving 269.5 grams of ethyl cellulose 100 cps, 196.0 grams of hydroxypropyl cellulose EFX, and 24.5 grams of Myrj 52 in 6510 grams of anhydrous ethanol SDA3A with stirring and warming. The ethyl cellulose had a molecular weight of approximately 220,000 and the hydroxypropyl cellulose had a molecular weight of approximately 80,000. The solution was allowed to stand at ambient temperature for several days. This formed the membrane subcoat solution.

The smooth tablets from above were mixed into a bed of placebo tablets weighing 1.2 kg and the resulting mixed bed was charged into a Vector LDCS pan coater fitted with a 14-inch diameter coating pan. The membrane subcoat solution was then sprayed onto the bed of tablets in the coater in a current of warm air. The coating solution was stirred continuously during the process. The solution was applied in this manner until approximately 5.5 mils of coating was accumulated on each drug tablet.

Then, 175 grams of cellulose acetate 398-10 and 75 grams of Lutrol F68 were dissolved in 4,750 grams of acetone with warming and stirring. The cellulose acetate had an average acetyl content of approximately 39.8 weight percent and a molecular weight of approximately 40,000. This formed the membrane overcoat solution.

This membrane overcoat solution was applied to the bed of active and placebo cores in the LDCS pan coater until 5 mils of membrane overcoat accumulated on each drug tablet. The three-coated layers formed wall 20 of the present invention. A delivery port 60 was mechanically drilled through the three coating layers on the drug layer side of the tablets using a 40-mil diameter drill bit and drill press. The systems were then dried in a forced air oven at 40° C. to remove residual processing solvents.

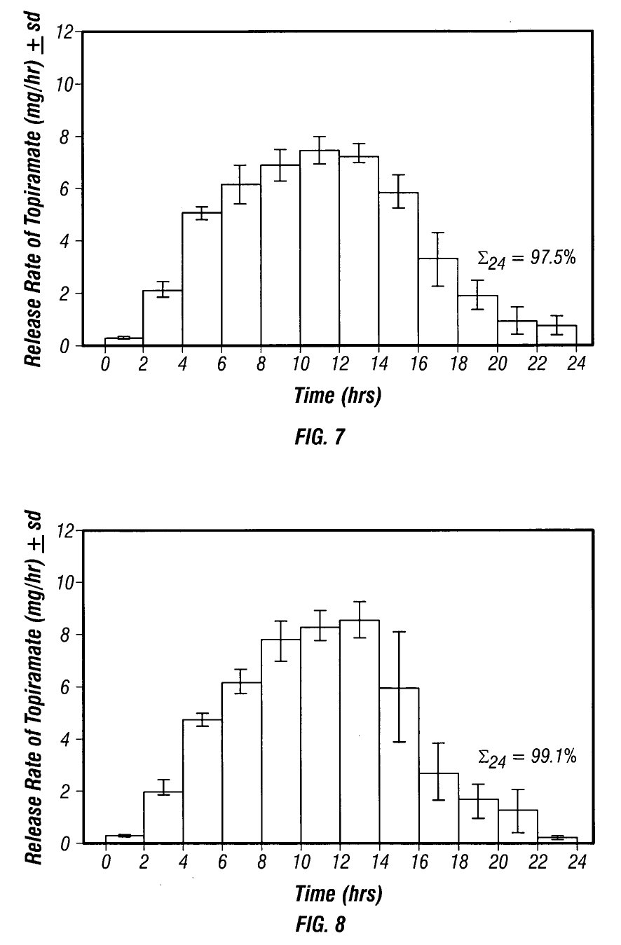

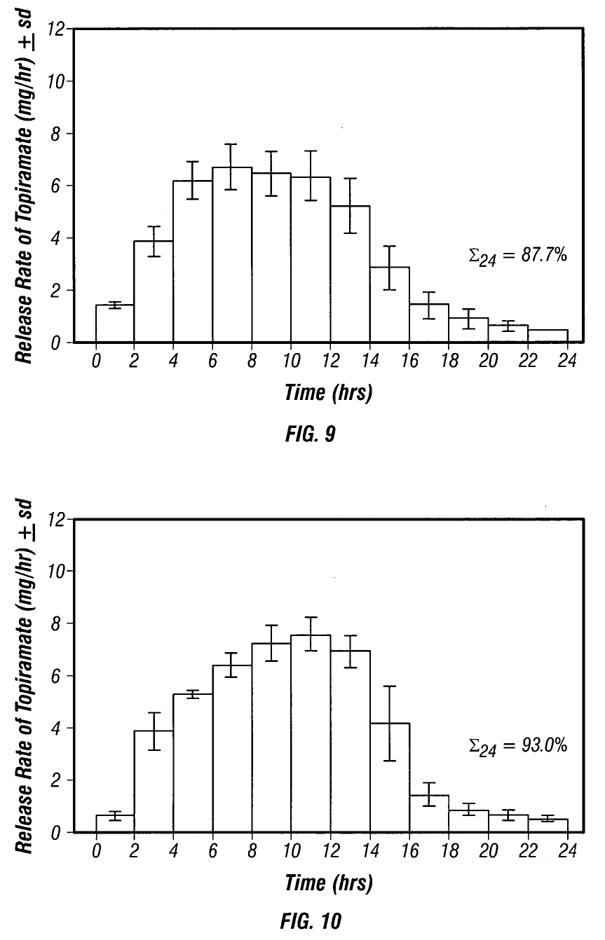

The resulting six systems were tested for release of drug in de-ionized water at 37° C. by sampling every 2 hours over duration of 24 hours. Drug release was monitored with refractive index chromatography. The resulting release pattern of drug is shown in FIG. 7. The drug 31 was delivered at an ascending release pattern for 12-14 hours. The time to deliver 90% of the 100 mg dose was approximately 18 hours. The cumulative delivery at 24 hours was 97.5%. The membranes were intact throughout the delivery pattern.

The systems were sufficiently small to easily be swallowed by a patient even with the high drug loading of 55% present in the drug layer 30.

Similar systems with expandable push layers were formulated with 55% drug in the drug layer, but without the solubilizing surfactant in an attempt to implement prior art technology but such systems of the prior art were not operational. These formulations representing the prior art did not solublize the drug and resulted in drug layers that could not be pumped. The membranes of these systems split open in situ during in vitro testing, dumping the bolus of drug in an uncontrolled fashion, due to the strain induced within the membrane by the swelling pressure generated by the expanding push layer pushing against the insoluble drug mass composition through the narrow 40-mil port.

### EXAMPLE 2

A drug core composition of 9.0 grams of micronized Lutrol F 127 was dry mixed with 16.5 grams of topiramate. The topiramate had a nominal particle size of 80 microns. Next, 3.45 grams Polyox N80 and 0.9 grams of polyvinyl pyrrolidone were sieved through minus 40 mesh and blended into the mixture. Then, 5 grams of anhydrous ethanol was added slowly with stirring to form a damp mass. The damp mass was passed through a # 16 mesh sieve and air dried overnight at ambient temperature. The resulting dried noodles were passed again through # 16 mesh sieve. Then, 150 mg of magnesium stearate was passed through a # 60 mesh sieve over the dried granules and tumble mixed into the granules. The concentration of surfactant in this drug core composition granulation was 30 weight percent.

The expandable push layer granulation was prepared by passing 63.67 grams of Polyox 303, 30 grams of sodium chloride, and 5 grams of hydroxypropyl methyl cellulose through a # 40 mesh sieve and dry mixing to form a uniform blend. Then, 1.0 gram of ferric oxide red was passed though a #60 mesh sieve into the mixture. The resulting mixture was wet massed by slowly adding anhydrous ethyl alcohol SDA3A anhydrous with stirring to form a uniformly damp mass. The mass was passed through a # 20 mesh sieve, resulting in noodles that were dried at 40° C. in forced air overnight. The dried noodles were passed through a # 16 mesh sieve to form free-flowing granules. Finally, 25 mg of magnesium stearate and 8 mg of butylated hydroxytoluene were sieved through a # 80 mesh sieve into the granules and tumble mixed.

A portion of the drug core composition granulation weighing 182 mg was filled into a round 3/16-inch diameter die and lightly compressed with 3/16-inch concave punches. Then, 60 mg of the expandable push layer granulation was added to the drug layer and the two layers were laminated with a force of 800 pounds. Six tablets were made.

The tablets were coated as described in Example 1 with 5 mg of the smoothing coat, 5.4 mils of the subcoat membrane, and 5.7 mils of the overcoat membrane. One exit port of 40 mils diameter was drilled through the three coating layers and the systems were dried overnight at 40° C. in forced air.

The resulting systems were tested as described in Example 1. The release profile of topiramate is shown in FIG. 8. The systems released 99% of the drug over a 24-hour duration. The release rate is smoothly ascending in time during the first 14 hours where 76% of the drug is released. The system released approximately 90% of the drug over 19 hours. The final system is of the same size that is convenient and feasible for patients in need to swallow as described in Example 1.

### EXAMPLE 3

Systems are made as described in Example 2 but surfactant 33 comprises a blend of two solubilizing surfactants. The drug core composition granulation was made according to the procedures in Example 2 except the surfactant consists of 15 weight percent micronized Lutrol F 127 and 15 weight percent Myrj 52 substituted for 30 weight percent micronized Lutrol F127. The weighted average HLB value of the two surfactants yields an HLB value of 19.5 that is mid point between the two HLB values of the single surfactants.

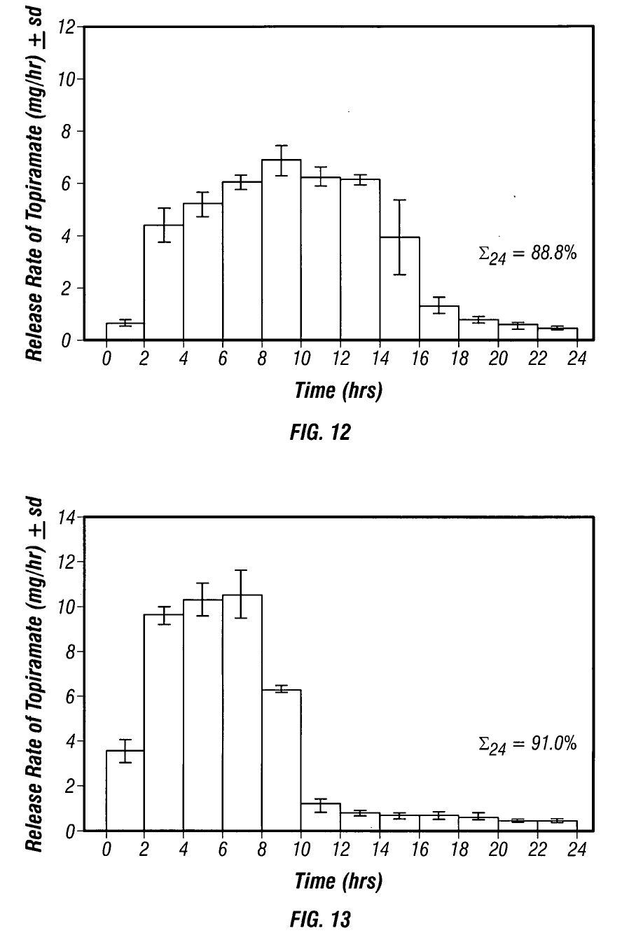

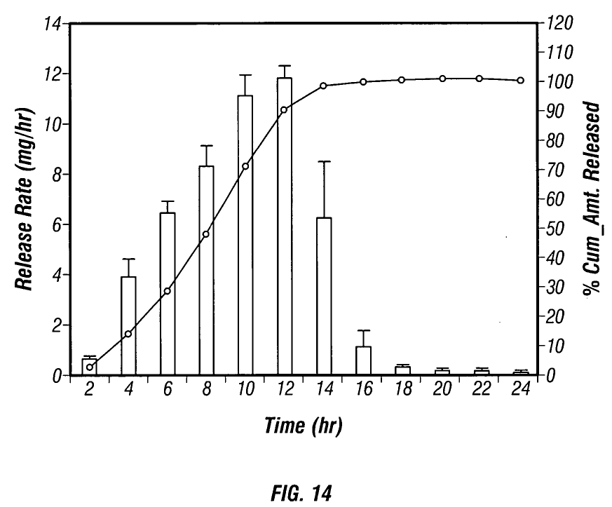

The delivery pattern of the resulting systems is shown in FIG. 12. The system delivers at essentially zero order rate between hour 2 and hour 14. The systems released 89% of the dose over 24 hours.

### EXAMPLE 4

Systems are made as described in Example 3 but with a larger weight of the expandable push layer. The expandable push layer weight is 90 mg substituted for the 60 mg weight of the systems in Example 3.

The delivery pattern of the resulting systems is shown in FIG. 13. The system delivers at an ascending release rate for about 12 hours, then the rate becomes descending. The amount of drug delivered over 24 hours is 93%.

### EXAMPLE 5

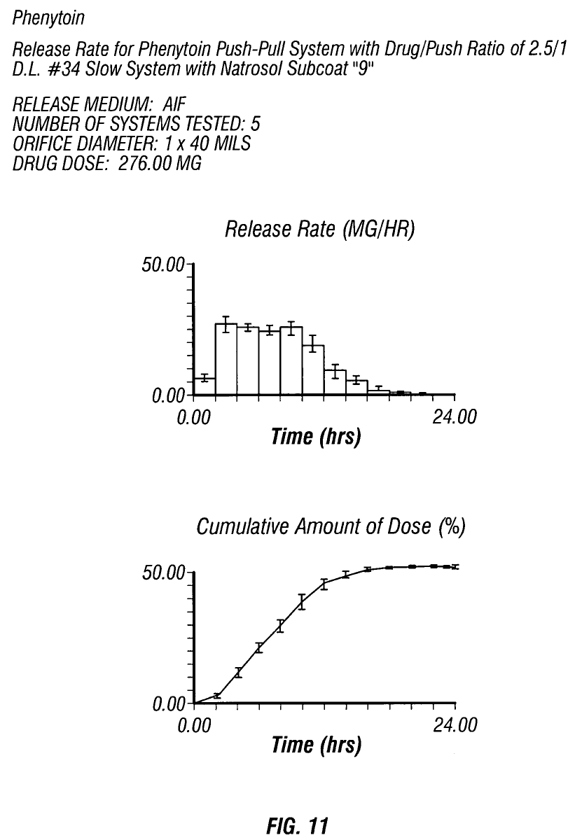

Capsule shaped tablet form, see FIG. 11.

### EXAMPLE 6

A drug composition, drug layer 30, was formed consisting of 30 wt % drug topiramate, 56 wt % surfactant Lutrol F127, 10 wt % carrier Polyox N-80 and 3 wt % PVPK2932 and 2 wt % Stearic acid by wet granulating with anhydrous ethanol.

A push composition consisting of 63.37 wt % Polyox 303 (7,000,000 molecular weight), 30 wt % NaCl, 5 wt % HPMC E5, 1 wt % Ferric Oxide, 0.5 wt % Mg Stearate and 0.08 wt % BHT was wet granulated with anhydrous ethanol.

Tablets with 333 mg of the drug core composition (100 mg topiramate) and 133 mg push composition were compressed using a 9/32″ longitudinally compressed tablet tooling. Total tablet (capsule shape) weight is 466 mg. The systems were coated, drilled, and dried according to the procedures described in Example 1. The systems were drilled and tested for release of drug, producing a zero order release pattern delivering the drug at steady rate of about 5.8 mg per hour over approximately 16 hours.

### EXAMPLE 7

A drug core composition containing 55 wt % drug phenytoin, 36.50 wt % carrier Polyox® N-80 and 3 wt % PVP K2932; 5 wt % surfactant MYRJ 52S; and 0.50 wt % magnesium stearate was wet granulated with anhydrous ethanol.

A push composition with the same composition as in Example 6 was wet granulated with anhydrous ethanol.

Tablets with 502 mg of drug core composition and 201 mg of push composition were compressed using a 9/32″ LCT tooling to produce bilayer capsule-shaped tablets. These tablets were subcoated with 66 mg of 95/5 wt % HEC 250L/PEG 3350 and 47 mg semi-permeable membrane consisting of 85/15 wt % of cellulose acetate 398-10/PEG 3350. An orifice is drilled on the drug layer as delivery port. Systems were tested for drug release. FIG. 11 shows the release profile of these systems. The systems release phenytoin at zero order rate of approximately 24 mg per hour over a duration of approximately 10 hours.

### EXAMPLE 8

### Topiramate Capsule Shaped Trilayer 100 mg System

A dosage form adapted, designed and shaped as an osmotic drug delivery device is manufactured as follows beginning with the drug layer. First, 3000 g of topiramate, 2520 g of polyethylene oxide with average molecular weight of 200,000 and 3630 g of poloxamer 407 (Lutrol F127) having an average molecular weight of 12,000 are added to a fluid bed granulator bowl. Next two separate binder solutions, the poloxamer binder solution and the polyvinylpyrrolidone identified as K29-32 having an average molecular weight of 40,000 binder solution are prepared by dissolving 540 g of the same poloxamer 407 (Lutrol F127 ) in 4860 g of water and 495 g of the same polyvinylpyrrolidone in 2805 of water, respectively. The dry materials are fluid bed granulated by first spraying with 2700 g of the poloxamer binder solution and followed by spraying 2000 g of the polyvinylpyrrolidone binder solution. Next, the wet granulation is dried in the granulator to an acceptable moisture content, and sized using by passing through a 7-mesh screen. Next, the granulation is transferred to a blender and mixed with 5 g of butylated hydroxytoluene as an antioxidant and lubricated with 200 g of stearic acid and 75 g of magnesium stearate.

Next, the drug layer is prepared as follows: 4000 g of topiramate, 213 g of polyethylene oxide with average molecular weight of 200,000, 4840 g of poloxamer 407 (Lutrol F127 ) having an average molecular weight of 12,000 and 10 g of ferric oxide, black are added to a fluid bed granulator bowl. Next, two separate binder solutions, the poloxamer binder solution and the polyvinylpyrrolidone identified as K29-32 having an average molecular weight of 40,000 binder solution are prepared by dissolving 720 g of the same poloxamer 407 in 6480 g of water and 495 g of the same polyvinylpyrrolidone in 2805 of water, respectively. The dry materials are fluid bed granulated by first spraying with 3600 g of the poloxamer binder solution and followed by spraying 2000 g of the polyvinylpyrrolidone binder solution. Next, the wet granulation is dried in the granulator to an acceptable moisture content, and sized using by passing through a 7-mesh screen. Next, the granulation is transferred to a blender and mixed with 2 g of butylated hydroxytoluene as an antioxidant and lubricated with 200 g of stearic acid and 75 g of magnesium stearate.

Next, a push composition is prepared as follows: first, a binder solution is prepared. 7.5 kg of polyvinylpyrrolidone identified as K29-32 having an average molecular weight of 40,000 is dissolved in 50.2 kg of water. Then, 37.5 kg of sodium chloride and 0.5 kg of ferric oxide are sized using a Quadro Comil with a 21-mesh screen. Then, the screened materials and 80.4 kg of Polyethylene oxide (approximately 7,000,000 molecular weight) are added to a fluid bed granulator bowl. The dry materials are fluidized and mixed while 48.1 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 63 g of butylated hydroxytoluene and lubricated with 310 g stearic acid.

Next, the topiramate drug compositions (first drug layer and second drug layer) and the push composition are compressed into trilayer tablets on multilayer Korsch press. First, 120 mg of the topiramate first drug layer composition is added to the die cavity and pre-compressed, then, 160 mg of the topiramate second drug layer composition is added to the die cavity and pre-compressed again, and finally, the push composition is added to achieve the total system weight of 480 mg and the layers are pressed into a ¼″ diameter, capsule shaped, deep concave, trilayer arrangement.

The trilayer arrangements are coated with bilayer polymer membrane laminate in which the first coating layer is a rigid yet water permeable laminate and the second coating layer is a semi-permeable membrane laminate. The first membrane laminate composition comprises 55% ethylcellulose, 45% hydroxylpropyl cellulose and 5% polyoxyl 40 stearate (PEG 40 stearate or Myrj 52S). The membrane-forming composition is dissolved in 100% ethyl alcohol to make a 7% solids solution. The membrane-forming composition is sprayed onto and around the Trilayer arrangements in a 10 kg scale pan coater until approximately 45 mg of membrane is applied to each tablet.

Next, the trilayer arrangements coated with the first membrane laminate are coated with the semi-permeable membrane. The membrane forming composition comprises 80% cellulose acetate having a 39.8% acetyl content and 20% poloxamer 188 (Pluronic F68 or Lutrol F68). The membrane-forming composition is dissolved in 100% acetone solvent to make a 5% solids solution. The forming-forming composition is sprayed onto and around the trilayer arrangements in a pan coater until approximately 35 mg of membrane is applied to each tablet.

Next, one 40 mil (1 mm) exit passageway is laser drilled through the bilayer membrane laminate to connect the drug layer with the exterior of the dosage system. The residual solvent is removed by drying for 72 hours at 40 C and ambient humidity.

Next, the drilled and dried systems are color overcoated. The color overcoat is a 12% solids suspension of Opadry in water. The color overcoat suspension is sprayed onto the trilayer systems until an average wet coated weight of approximately 25 mg per system is achieved.

Next, the color-overcoated systems are clear coated. The clear coat is a 5% solids solution of Opadry in water. The clear coat solution is sprayed onto the color coated cores until an average wet coated weight of approximately 10 mg per system is achieved.

The dosage form produced by this manufacture is designed to deliver 100 mg of topiramate in an ascending manner at certain controlled-delivery rate from the core containing the first drug layer of 30% topiramate, 25.2% polyethylene oxide possessing a 200,000 molecular weight, 39% poloxamer 407 (Lutrol F127 ), 3% polyvinylpyrrolidone possessing a 40,000 molecular weight, 0.05% butylated hydroxytoluene, 2% stearic acid and 0.75% magnesium stearate, and the second drug layer of 40% topiramate, 2.13% polyethylene oxide possessing a 200,000 molecular weight, 52% poloxamer 407 (Lutrol F127 ), 3% polyvinylpyrrolidone possessing a 40,000 molecular weight, 0.1% black ferric oxide, 0.05% butylated hydroxytoluene, 2% stearic acid and 0.75% magnesium stearate. The push composition is comprised 64.3% polyethylene oxide comprising a 7,000,000 molecular weight, 30% sodium chloride, 5% polyvinylpyrrolidone possessing an average molecular weight of 40,000, 0.4% ferric oxide, 0.05% butylated hydroxytoluene, and 0.25% stearic acid. The bilayer membrane laminate in which the first membrane layer is comprised of 55% ethylcellulose, 45% hydroxylpropyl cellulose and 5% polyoxyl 40 stearate (PEG 40 stearate or Myrj 52S), and the second membrane laminate is a semi-permeable wall which is comprised of 80% cellulose acetate of 39.8% acetyl content and 20% poloxamer 188 (Pluronic F68 or Lutrol F68). The dosage form comprises one passageway, 40 mils (1 mm) on the center of the drug side. The final dosage form contains a color overcoat and a clear overcoat and the time to achieve 90% of drug release in an ascending manner is approximately 16 hours.

### EXAMPLE 9

### Topiramate Capsule Shaped Trilayer 12.5 mg System

A dosage form adapted, designed and shaped as an osmotic drug delivery device is manufactured as follows beginning with the first drug layer. First, 4 g of topiramate, 40 g of polyethylene oxide with average molecular weight of 200,000, 4 g of poloxamer 407 (Lutrol F127) having an average molecular weight of 12,000 and 1.5 g of polyvinylpyrrolidone identified as K29-32 having an average molecular weight of 40,000 are added to a beaker or mixing bowl. Next, the dry materials are mixed for 60 seconds. Then 16 mL of denatured anhydrous alcohol was slowly added to blended materials with continuous mixing for approximately 2 minutes. Next, the freshly prepared wet granulation was allowed to dry at room temperature for approximately 16 hours, and passed through a 16-mesh screen. Next, the granulation were transferred to an appropriate container, mixed and lubricated with 0.5 g of stearic acid.

Next, the second drug layer is prepared as follows: 6 g of topiramate, 35.95 g of polyethylene oxide with average molecular weight of 200,000, 6 g of poloxamer 407 (Lutrol F127 ) having an average molecular weight of 12,000, 1.5 g of polyvinylpyrrolidone identified as K29-32 having an average molecular weight of 40,000 and 0.05 g of ferric oxide are added to a beaker or mixing bowl. Next, the dry materials are mixed for 60 seconds. Then 16 mL of denatured anhydrous alcohol was slowly added to blended materials with continuous mixing for approximately 2 minutes. Next, the freshly prepared wet granulation was allowed to dry at room temperature for approximately 16 hours, and passed through a 16-mesh screen. Next, the granulation were transferred to an appropriate container, mixed and lubricated with 0.5 g of stearic acid.

Next, a push composition is prepared as follows: first, a binder solution is prepared. 7.5 kg of polyvinylpyrrolidone identified as K29-32 having an average molecular weight of 40,000 is dissolved in 50.2 kg of water. Then, 37.5 kg of sodium chloride and 0.5 kg of ferric oxide are sized using a Quadro Comil with a 21-mesh screen. Then, the screened materials and 80.4 kg of Polyethylene oxide (approximately 7,000,000 molecular weight) are added to a fluid bed granulator bowl. The dry materials are fluidized and mixed while 48.1 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 63 g of butylated hydroxytoluene and lubricated with 310 g stearic acid.

Next, the topiramate drug compositions (first drug layer and second drug layer) and the push composition are compressed into trilayer tablets on the Carver Tablet Press. First, 56 mg of the topiramate first drug layer composition is added to the die cavity and pre-compressed, then, 67 mg of the topiramate second drug layer composition is added to the die cavity and pre-compressed again, and finally, the push composition is added to achieve the total system weight of 211 mg and the layers are pressed into a 3/16″ diameter capsule, deep concave, trilayer arrangement.

The trilayer arrangements are coated with bilayer polymer membrane laminate in which the first coating layer is a rigid yet water permeable laminate and the second coating layer is a semi-permeable membrane laminate. The coating is performed on a 10 kg scale pan coater by spike-loading the topiramate trilayer systems with the placebo tablets. The first membrane laminate composition comprises 55% ethylcellulose, 45% hydroxylpropyl cellulose and 5% polyoxyl 40 stearate (PEG 40 stearate or Myrj 52S). The membrane-forming composition is dissolved in 100% ethyl alcohol to make a 7% solids solution. The membrane-forming composition is sprayed onto and around the Trilayer arrangements in a pan coater until approximately 30 mg of membrane is applied to each tablet.

Next, the trilayer arrangements coated with the first membrane laminate are coated with the semi-permeable membrane. The membrane forming composition comprises 80% cellulose acetate having a 39.8% acetyl content and 20% poloxamer 188 (Pluronic F68 or Lutrol F68). The membrane-forming composition is dissolved in 100% acetone solvent to make a 5% solids solution. The forming-forming composition is sprayed onto and around the trilayer arrangements in a pan coater until approximately 25 mg of membrane is applied to each tablet.

Next, one 30 mil (0.76 mm) exit passageway is laser drilled through the bilayer membrane laminate to connect the drug layer with the exterior of the dosage system. The residual solvent is removed by drying for 72 hours at 40 C and ambient humidity.

Next, the drilled and dried systems are color overcoated. The color overcoat is a 12% solids suspension of Opadry in water. The color overcoat suspension is sprayed onto the trilayer systems until an average wet coated weight of approximately 15 mg per system is achieved.

The dosage form produced by this manufacture is designed to deliver 12.5 mg of topiramate in an ascending manner at certain controlled-delivery rate from the core containing the first drug layer of 8% topiramate, 80% polyethylene oxide possessing a 200,000 molecular weight, 8% poloxamer 407 (Lutrol F127 ), 3% polyvinylpyrrolidone possessing a 40,000 molecular weight and 1% stearic acid, and the second drug layer of 12% topiramate, 71.9% polyethylene oxide possessing a 200,000 molecular weight, 12% poloxamer 407 (Lutrol F127 ), 3% polyvinylpyrrolidone possessing a 40,000 molecular weight, 0.1% ferric oxide and 1% stearic acid. The push composition is comprised 64.3% polyethylene oxide comprising a 7,000,000 molecular weight, 30% sodium chloride, 5% polyvinylpyrrolidone possessing an average molecular weight of 40,000, 0.4% ferric oxide, 0.05% butylated hydroxytoluene, and 0.25% stearic acid. The bilayer membrane laminate in which the first membrane layer is comprised of 55% ethylcellulose, 45% hydroxylpropyl cellulose and 5% polyoxyl 40 stearate (PEG 40 stearate or Myrj 52S), and the second membrane laminate is a semi-permeable wall which is comprised of 80% cellulose acetate of 39.8% acetyl content and 20% poloxamer 188 (Pluronic F68 or Lutrol F68). The dosage form comprises one passageway, 30 mils (0.76 mm) on the center of the drug side. The final dosage form could contain a color overcoat and a clear overcoat and the time to achieve 90% of the drug release in an ascending manner is approximately 16 hours.

### EXAMPLE 10

### Topiramate Capsule Shaped Bilayer 100 mg System

A dosage form adapted, designed and shaped as an osmotic drug delivery device is manufactured as follows: First, 2880 g of topiramate, 958 g of polyethylene oxide with average molecular weight of 200,000 and 4980 g of poloxamer 407 (Lutrol F127 ) having an average molecular weight of 12,000 are added to a fluid bed granulator bowl. Next two separate binder solutions, the poloxamer binder solution and the polyvinylpyrrolidone identified as K29-32 having an average molecular weight of 40,000 binder solution are prepared by dissolving 500 g of the same poloxamer 407 (Lutrol F127 ) in 4500 g of water and 750 g of the same polyvinylpyrrolidone in 4250 of water, respectively. The dry materials are fluid bed granulated by first spraying with 3780 g of the poloxamer binder solution and followed by spraying 3333 g of the polyvinylpyrrolidone binder solution. Next, the wet granulation is dried in the granulator to an acceptable moisture content, and sized using by passing through a 7-mesh screen. Next, the granulation is transferred to a blender and mixed with 2 g of butylated hydroxytoluene as an antioxidant and lubricated with 200 g of stearic acid and 100 g of magnesium stearate.

Next, a push composition is prepared as follows: first, a binder solution is prepared. 7.5 kg of polyvinylpyrrolidone identified as K29-32 having an average molecular weight of 40,000 is dissolved in 50.2 kg of water. Then, 37.5 kg of sodium chloride and 0.5 kg of ferric oxide are sized using a Quadro Comil with a 21-mesh screen. Then, the screened materials and 80.4 kg of Polyethylene oxide (approximately 7,000,000 molecular weight) are added to a fluid bed granulator bowl. The dry materials are fluidized and mixed while 48.1 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 63 g of butylated hydroxytoluene and lubricated with 310 g stearic acid.

Next, the topiramate drug composition and the push composition are compressed into bilayer tablets on multilayer Korsch press. First, 278 mg of the topiramate composition is added to the die cavity and pre-compressed, then, the push composition is added to achieve the total system weight of 463 mg and the layers are pressed into a 15/64″ diameter, capsule shaped, deep concave, bilayer arrangement.

The bilayer arrangements are coated with bilayer polymer membrane laminate in which the first coating layer is a rigid yet water permeable laminate and the second coating layer is a semi-permeable membrane laminate. The first membrane laminate composition comprises 55% ethylcellulose, 45% hydroxylpropyl cellulose and 5% polyoxyl 40 stearate (PEG 40 stearate or Myrj 52S). The membrane-forming composition is dissolved in 100% ethyl alcohol to make a 7% solids solution. The membrane-forming composition is sprayed onto and around the arrangements in a pan coater until approximately 38 mg of membrane is applied to each tablet.

Next, the trilayer arrangements coated with the first membrane laminate are coated with the semi-permeable membrane. The membrane forming composition comprises 80% cellulose acetate having a 39.8% acetyl content and 20% poloxamer 188 (Pluronic F68 or Lutrol F68). The membrane-forming composition is dissolved in 100% acetone solvent to make a 5% solids solution. The forming-forming composition is sprayed onto and around the arrangements in a pan coater until approximately 30 mg of membrane is applied to each tablet.

Next, one 45 mil (1.14 mm) exit passageway is laser drilled through the bilayer membrane laminate to connect the drug layer with the exterior of the dosage system. The residual solvent is removed by drying for 72 hours at 40 C and ambient humidity.

Next, the drilled and dried systems are coated with an immediate release drug overcoat. The drug overcoat is a 13% solids aqueous solution containing 780 g of topiramate, 312 g of copovidone (Kollidone VA 64) and 208 g of hydroxypropyl methycellulose possessing an average molecular weight of 11,200. The drug overcoat solution is sprayed onto the dried coated cores until an average wet coated weight of approximately 33 mg per system is achieved.

Next, the drug-over coated systems are color over coated. The color overcoat is a 12% solids suspension of Ovary in water. The color overcoat suspension is sprayed onto the drug over coated systems until an average wet coated weight of approximately 25 mg per system is achieved.

Next, the color-over coated systems are clear coated. The clear coat is a 5% solids solution of Opadry in water. The clear coat solution is sprayed onto the color coated cores until an average wet coated weight of approximately 25 mg per system is achieved.

The dosage form produced by this manufacture is designed to deliver 20 mg of topiramate as an immediate release from an overcoat comprised of 60% topiramate, 24% copovidone and 16% hydroxypropyl methylcellulose followed by the controlled delivery of 80 mg of topiramate from the core containing 28.8% topiramate, 9.58% polyethylene oxide possessing a 200,000 molecular weight, 53.6% poloxamer 407 (Lutrol F127 ), 5% polyvinylpyrrolidone possessing a 40,000 molecular weight, 0.02% butylated hydroxytoluene, 2% stearic acid and 1% magnesium Stearate. The push composition is comprised 64.3% polyethylene oxide comprising a 7,000,000 molecular weight, 30% sodium chloride, 5% polyvinylpyrrolidone possessing an average molecular weight of 40,000, 0.4% ferric oxide, 0.05% butylated hydroxytoluene, and 0.25% stearic acid. The bilayer membrane laminate in which the first membrane layer is comprised of 55% ethylcellulose, 45% hydroxylpropyl cellulose and 5% polyoxyl 40 stearate (PEG 40 stearate or Myrj 52S), and the second membrane laminate is a semi-permeable wall which is comprised of 80% cellulose acetate of 39.8% acetyl content and 20% poloxamer 188 (Pluronic F68 or Lutrol F68). The dosage form comprises one passageway, 45 mils (1.14 mm) on the center of the drug side. The final dosage form contains a color overcoat and a clear overcoat and has a mean release rate of 6 mg topiramate per hour releasing in zero-order manner.

### DISCLOSURE FOR USING THE INVENTION

The invention also concerns a method for administering 1 ug to 750 mg of therapeutic agent to a patient in need of therapy. The method, in one administration, comprises admitting orally into the patient a therapeutic agent or its salt that is administered from a therapeutic composition, 5 mg to 500 mg of a structural polymer carrier having a 100,000 to 7 million molecular weight, and 5 to 600 mg of a surfactant having an HLB identified by drug solubility studies, which composition provides therapy over an extended period of time.

The invention provides methods for administering therapeutic agents to a patient, and methods for producing a plasma concentration of therapeutic agents. The method of the invention provides for admitting orally to a patient a dosage form that administers at a controlled rate, over a continuous time up to 24 hours, drug for its intended therapy. The method also comprises administering orally to a patient a therapeutic dose of therapeutic agent from a single dosage form that administers the agent over 24 hours.

Inasmuch as the foregoing specification comprises disclosed embodiments, it is understood what variations and modifications may be made herein, in accordance with the principles disclosed, without departing from the invention.

We claim:

1. A controlled release therapeutic composition comprising a low solubility therapeutic agent, a structural polymer carrier and a solubilizing surfactant.

2. A controlled release therapeutic composition comprising a low solubility therapeutic agent, a structural polymer carrier and a solubilizing surfactant adapted to release a high dose of the therapeutic agent.

3. The composition of claim 2 wherein the high dose of therapeutic agent is between about 1 μg and 750 mg of the therapeutic agent.

4. The composition of claim 2 wherein the high dose of therapeutic agent is between about 10 mg and about 250 mg of the therapeutic agent.

5. The composition of claim 2 wherein the high dose of therapeutic agent is between about 25 mg and about 400 mg of the therapeutic agent.

6. The composition of claim 2 wherein the therapeutic agent has solubility that is between about 1 μg/ml and about 100 mg/ml.

7. The composition of claim 2 wherein the therapeutic agent has solubility that is between about 1 μg/ml and about 50 mg/ml.

8. The composition of claim 2 wherein the amount of structural polymer is between about 1% and 80% by weight of the composition.

9. The composition of claim 2 wherein the amount of structural polymer is between about 5% and 50% by weight of the composition.

10. The composition of claim 2 wherein the amount of structural polymer is between about 5% and 15% by weight of the composition.

11. The composition of claim 2 wherein the structural polymer is polyethylene oxide of about 100,000 to 200,000 molecular weight.

12. The composition of claim 2 wherein the solubilizing surfactant is selected from the group consisting of polyoxyl 40 stearate, polyoxyl 50 stearate, poloxamers, and a:b:a triblock copolymers of ethylene oxide:propylene oxide:ethylene oxide.

13. The composition of claim 2 wherein the amount of solubilizing surfactant is between about 5% and 50% by weight of the composition.

14. The composition of claim 2 wherein the amount of solubilizing surfactant is between about 5% and 40% by weight of the composition.

15. A composition comprising a low solubility therapeutic agent, a structural polymer and a solubilizing surfactant adapted to release the therapeutic agent over a prolonged period of time.

16. A composition comprising a low solubility therapeutic agent, a structural polymer and a solubilizing surfactant wherein the composition is a solid.

17. A controlled release pharmaceutical composition comprising a low solubility therapeutic agent, a structural polymer and a solubilizing surfactant adapted to increase the solubility of the therapeutic agent.

18. A dosage form for controlled release of a therapeutic composition comprising a low solubility therapeutic agent, a structural polymer and a solubilizing surfactant.

19. The dosage form of claim 18 wherein the dosage form is a matrix system.

20. The dosage form of claim 18 wherein the dosage form is an osmotic system.

21. The dosage form of claim 18 wherein the dosage form is adapted to be administered once a day.

22. The dosage form of claim 18, which is adapted to release a high dose of the therapeutic agent.

23. The dosage form of claim 22 wherein the high dose of the therapeutic agent is between about 20% and about 90% by weight of the therapeutic composition.

24. The dosage form of claim 22 wherein the high dose of the therapeutic agent is between about 30% and about 40% by weight of the therapeutic composition.

25. A controlled release oral dosage form for once-a-day administration of a therapeutic agent comprising:
a. A core which comprises:
i. a low solubility therapeutic agent;
ii. a structural polymer;
iii. a solubilizing surfactant;
b. a semipermeable membrane surrounding the core; and
c. an exit orifice through the semipermeable membrane which communicates with the core so as to allow release of the therapeutic agent to the environment;
wherein the dosage form releases the therapeutic agent over a prolonged period of time.

26. The controlled release oral dosage form of claim 25 adapted to release the therapeutic agent at a substantially zero order release rate.

27. The controlled release oral dosage form of claim 25 adapted to release the therapeutic agent at a substantially ascending release rate.

28. A method for delivering high doses of low solubility therapeutic agents comprising orally administering the dosage form of claim 25 to a subject.

29. A method for enhancing the bioavailability of a therapeutic agent comprising orally administering the dosage form of claim 25 to a subject.
